(19) United States
(12) Patent Application Publication
(10) Pub. No.: US 2025/0325802 A1
(43) Pub. Date: Oct. 23, 2025

(54) ELECTRODE LEAD ASSEMBLY

(71) Applicant: Saluda Medical Pty Limited, Macquarie Park (AU)

(72) Inventors: Peter Scott Vallack Single, Macquarie Park (AU); Dean Michael Karantonis, Macquarie Park (AU); David Martin Mugan, Macquarie Park (AU); Daniel John Parker, Macquarie Park (AU)

(21) Appl. No.: 19/186,410

(22) Filed: Apr. 22, 2025

(30) Foreign Application Priority Data
Apr. 23, 2024 (AU) 2024901146

(51) Int. Cl. A61N 1/05; A61N 1/36

(52) U.S. Cl. CPC A61N 1/0553; A61N 1/0556; A61N 1/36071; A61N 1/36139

(57) ABSTRACT

Disclosed is an electrode lead assembly comprising an electrode lead body and a plurality of electrodes arranged along a length of the body. The plurality of electrodes comprising a first group of electrodes and a second group of electrodes, wherein adjacent pairs of electrodes in each group of electrodes have a respective electrode pitch. The first group and the second group of electrodes each comprise at least one electrode configurable as a stimulus electrode for delivering stimulation to a neural target and at least one electrode configurable as a measurement electrode for measuring a response evoked by the delivered stimulation. The first group of electrodes is separated from the second group of electrodes by an electrode-free lead portion, the electrode-free lead portion having a length that is greater than each electrode pitch of the first group of electrodes and greater than each electrode pitch of the second group of electrodes.

The present application claims priority from Australian Provisional Patent Application No. 2024901146 filed on 23 Apr. 2024, the contents of which are incorporated herein by reference in their entirety.

### TECHNICAL FIELD

The present invention relates to leads including electrodes for providing stimulus to generate a neural response, and in particular to an electrode lead assembly to provide stimulus and measure a neural response to stimulus, systems including the electrode lead assembly, and methods for using the electrode lead assembly.

### BACKGROUND OF THE INVENTION

There is a range of situations in which it is desirable to apply neural stimuli in order to alter neural function, a process known as neuromodulation. For example, neuromodulation is used to treat a variety of disorders including chronic neuropathic pain, incontinence, stroke rehabilitation, spinal cord injury, movement disorders including Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either inhibitory or excitatory effects on neural networks. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode assembly is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Action potentials propagating along A B (A-beta) fibres being stimulated in this way may inhibit the transmission of pain from a region of the body innervated by the target neural fibres (the dermatome) to the brain. To sustain the pain relief effects, stimuli are applied repeatedly, for example at a stimulus frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aß fibres or recruitment of undesired fibre classes. When recruitment is too large, Aβ fibres may produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), either of which can significantly alter the neural recruitment arising from a given stimulus, and therefore the therapeutic range. There is room in the epidural space for the electrode assembly to move, and such assembly movement from migration or posture change alters the electrode-to-cord distance and thus the recruitment efficacy of a given stimulus. Moreover, the spinal cord itself can move within the cerebrospinal fluid (CSF) with respect to the dura. During postural changes, the amount of CSF or the distance between the spinal cord and the electrode can change significantly. This effect is so large that postural changes alone can cause a previously comfortable and effective stimulus regime to become either ineffectual or painful.

Another control problem facing neuromodulation devices of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable devices, increased power consumption results in more frequent charging and, given that batteries only permit a limited number of charging cycles, ultimately this reduces the implanted lifetime of the device.

Attempts have been made to address such problems by way of feedback or closed-loop control, such as using the methods set forth in International Patent Publication No. WO2012/155188 by the present applicant, the contents of which is incorporated herein by reference. Feedback control seeks to compensate for relative nerve/electrode movement by controlling the intensity of the delivered stimuli so as to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to the target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be sensed by a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the contents of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the sensed signal as crosstalk of that magnitude. Moreover, stimulus generally results in electrode artefact, which may manifest in the sensed signal as a decaying output of the order of several millivolts after the end of the stimulus. As the neural response can be contemporaneous with the stimulus crosstalk or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result in a time-decaying artefact waveform of positive or negative polarity, their identification and elimination can be laborious.

Evoked neural responses are less difficult to measure when they appear later in time than the artefact, or when the signal-to-noise ratio is sufficiently high. The artefact is often restricted to a time of 1-2 ms after the stimulus and so, provided the neural response is measured after this time window, a neural response measurement can be more easily obtained. This is the case in surgical monitoring where there are large distances (e.g. more than 12 cm for nerves conducting at 60 ms-1) between the stimulus and measurement electrodes so that the propagation time from the stimulus site to the measurement electrodes exceeds 2 ms, which is longer than the typical duration of stimulus artefact.

However, to characterize the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity. In such situations the measurement process must overcome artefact directly.

The difficulty of this problem is further exacerbated when attempting to implement ECAP detection in an implanted device. Typical implanted devices have a power budget that permits a limited number, for example in the hundreds or low thousands, of processor instructions per stimulus, in order to maintain a desired battery lifetime. Accordingly, if a ECAP detector for an implanted device is to be used regularly (e.g. once a second), then care must be taken that the detector should consume only a small fraction of the power budget.

A functional feedback loop can also produce useful data for live operation or post-analysis, such as observed neural response intensity and applied stimulus intensity. However, device operation at tens of Hz over the course of hours or days quickly produces large volumes of such data which far exceed an implanted device's data storage capacities.

The design and configuration of the electrode lead assembly can impact the efficacy of the neuromodulation provided to a patient. Additionally, the design and configuration of the electrode lead assembly can impact the manufacturing and operational costs associated with the lead. Accordingly, it is desirable to determine a preferable design and configuration of an electrode lead assembly.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of the present disclosure.

### SUMMARY OF THE INVENTION

According to a first aspect of the present technology, there is provided an electrode lead assembly comprising an electrode lead body and a plurality of electrodes arranged along a length of the electrode lead body. The plurality of electrodes comprise a first group of electrodes and a second group of electrodes, each group of electrodes comprising two or more electrodes, wherein adjacent pairs of electrodes in each group of electrodes have a respective electrode pitch. The first group of electrodes and the second group of electrodes each comprise at least one electrode configurable as a stimulus electrode for delivering stimulation to a neural target, and at least one electrode configurable as a measurement electrode for measuring a response evoked by the delivered stimulation. The first group of electrodes is separated from the second group of electrodes by an electrode-free lead portion, the electrode-free lead portion having a length that is greater than each electrode pitch of the first group of electrodes and greater than each electrode pitch of the second group of electrodes.

The respective electrode pitches of the first and second groups of electrodes may be the same.

In some implementations, the plurality of electrodes further comprises a third group of electrodes. In some implementations, each of the first group of electrodes, the second group of electrodes and the third group of electrodes comprises three electrodes. In some implementations, each of the first group of electrodes, the second group of electrodes and the third group of electrodes are configurable to provide tripolar stimulation.

In some implementations, each of the first group of electrodes, the second group of electrodes and the third group of electrodes comprises at least two electrodes configurable as measurement electrodes. In some implementations, the second group of electrodes is separated from the third group of electrodes by a second electrode-free lead portion, the second electrode-free lead portion having a length that is greater than an electrode pitch of the second group of electrodes and greater than an electrode pitch of the third group of electrodes.

In some implementations, the first group of electrodes comprises at least two slim electrodes. In some implementations, at least one of the first group of electrodes, the second group of electrodes or the third group of electrodes comprises at least two slim electrodes. In some implementations, at least one electrode of the at least two slim electrodes has a length of at least 1.5 millimetres, or at least about 1.5 millimetres. In some implementations, at least one electrode of the at least two slim electrodes has a length no greater than 2 millimetres, or no greater than about 2 millimetres. In some implementations, the two slim electrodes are adjacent electrodes, and an electrode pitch between the two slim electrodes is equal to or greater than 2 millimetres (or about 2 millimetres) and equal to or less than 4 millimetres (or about 4 millimetres).

In some implementations, each electrode of the plurality of electrodes comprises a ring electrode. In some implementations, the electrode lead assembly is configured to be electrically connected to an implantable stimulation device comprising stimulation circuitry and measurement circuitry.

In some implementations, at least two electrodes are configured as measurement electrodes, and the two measurement electrodes have a pitch of 1.5 to 2 times a pitch of a pair of electrodes configured as stimulation electrodes. In some implementations, at least one group of electrodes has adjacent pairs of electrodes with a varying pitch. In some implementations the plurality of electrodes arranged along the electrode lead body comprise one or more middle electrodes, wherein the pitch for adjacent pairs of electrodes increases for adjacent pairs closer to the one or more middle electrodes. In some implementations a profile of the pitch variation is substantially linear. In some implementations a profile of the pitch variation is symmetrical about the one or more middle electrodes.

According to a further aspect of the present technology, there is provided an electrode lead assembly comprising an electrode lead body and a plurality of electrodes arranged along a length of the electrode lead body. The plurality of electrodes comprise a first group of electrodes and a second group of electrodes, each group of electrodes comprising two or more electrodes, wherein adjacent electrodes in each group of electrodes are separated by a respective electrode pitch. The first group of electrodes and the second group of electrodes each comprise at least one electrode configurable as a stimulus electrode for delivering stimulation to a neural target, and at least one electrode configurable as a measurement electrode for measuring a response evoked by the delivered stimulation. The electrode lead assembly may have more than two groups of electrodes.

There is preferably an electrode free portion between the first and second groups, or between other groups of electrodes.

Preferably, there is a varying (e.g. increasing, or decreasing) pitch for sequential pairings of adjacent electrodes in the first or second group. In one implementation, more proximally located pairs of adjacent electrodes have a smaller pitch than more distally located pairs of adjacent electrodes, for a most proximal group. For a most distal group of electrodes, located at a distal portion of the lead, the most distal group may have a varying pitch between sequential pairs of adjacent electrodes, so that more proximally located pairs of adjacent electrodes have a larger pitch than more distally located pairs of adjacent electrodes.

An increasing or a decreasing pitch of adjacent pairs of electrodes may increase or decrease, respectively, in a linear manner. For example, within a first group of electrodes, the most proximal pair of adjacent electrodes may have a pitch of x millimetres, the second most proximal pair of adjacent electrodes may have a pitch of x+1 millimetres, and the third most proximal pair of adjacent electrodes may have pitch of x+2 millimetres and so on. Similarly, within the second group, the most distal pair of adjacent electrodes may have a pitch of x millimetres, the second most distal pair of adjacent electrodes may have a pitch of x+1 millimetres, and the third most distal pair of adjacent electrodes may have pitch of x+2 millimetres and so on. The rate of increasing or decreasing may alternatively be non-linear.

In some implementations, there may be parallel rows of electrodes each row containing a plurality of electrode groups (e.g. two percutaneous leads may be configured to be implantable parallel and in-line with each other, or two rows of electrodes may be arranged on a paddle lead). The electrode locations may be offset, comparing one row of electrodes to another, to broaden selectivity and coverage of electrodes configured for stimulation or measuring.

There may be more than two groups of electrodes arranged in a similar manner, having increasing or decreasing pitches between sequential pairs of adjacent electrodes.

Preferably, the first group of electrodes is separated from the second group of electrodes by an electrode-free lead portion, the electrode-free lead portion having a length that is greater than the electrode pitch of the first group of electrodes and greater than the electrode pitch of the second group of electrodes.

Preferably, the first group of electrodes is separated from the second group of electrodes by an electrode-free lead portion, the electrode-free lead portion having a length that is about the same as or greater than the largest electrode pitch between adjacent electrodes in the first group of electrodes and about the same as or greater than the largest electrode pitch between adjacent electrodes of the second group of electrodes.

According to another aspect of the present technology, there is provided an implantable device for controllably stimulating a neural target, the device configurable to electrically couple to an electrode lead assembly described herein. The implantable device comprising stimulation circuitry, configurable to provide stimulation energy to one or more electrodes of the electrode lead assembly, measurement circuitry, configurable to measure a response evoked from the neural target by the stimulation energy and sensed by one or more electrodes of the electrode lead assembly, and an electrode selection module. The electrode selection module is configurable to select at least one first electrode from the plurality of electrodes of the electrode lead assembly and electrically couple the first electrode to the stimulation circuitry, and select at least one second electrode from the plurality of electrodes of the electrode lead assembly and electrically couple the second electrode to the measurement circuitry.

According to another aspect of the present technology, there is provided a system for controllable neural stimulation including an implantable pulse generator and an electrode lead assembly. The electrode lead assembly comprises a plurality of electrodes comprising a first group of electrodes and a second group of electrodes, each group of electrodes comprising two or more electrodes, wherein adjacent pairs of electrodes in each group of electrodes have a respective electrode pitch. The first group of electrodes and the second group of electrodes each comprise at least one electrode configurable as a stimulus electrode for delivering stimulation to a neural target, and at least one electrode configurable as a measurement electrode for measuring a response evoked by the delivered stimulation. The first group of electrodes is separated from the second group of electrodes by an electrode-free lead portion, the electrode-free lead portion having a length that is greater than each electrode pitch of the first group of electrodes and greater than each electrode pitch of the second group of electrodes. The implantable pulse generator comprises stimulation circuitry, configurable to provide stimulation energy to one or more electrodes of the electrode lead assembly, measurement circuitry, configurable to measure a response evoked from the neural target by the stimulation energy and sensed by one or more electrodes of the electrode lead assembly, and an electrode selection module. The electrode selection module is configurable to select at least one first electrode from the plurality of electrodes of the electrode lead assembly and electrically couple the first electrode to the stimulation circuitry, and select at least one second electrode from the plurality of electrodes of the electrode lead assembly and electrically couple the second electrode to the measurement circuitry.

According to another aspect of the present technology, there is provided a method of delivering neural stimuli to a neural pathway using an electrode lead assembly. The method comprises delivering, via a stimulus electrode of plurality of electrodes of the electrode lead assembly, a neural stimulus to the neural pathway in order to evoke a neural response from the neural pathway, measuring, via a measurement electrode of the plurality of electrodes of the electrode lead assembly, an intensity of a neural response evoked by the neural stimulus. The electrode lead assembly comprises a plurality of electrodes comprising a first group of electrodes and a second group of electrodes, each group of electrodes comprising two or more electrodes, wherein adjacent electrodes in each group of electrodes are separated by a respective electrode pitch. The first group of electrodes and the second group of electrodes each comprise at least one electrode configurable as a stimulus electrode for delivering stimulation to a neural target, and at least one electrode configurable as a measurement electrode for measuring a response evoked by the delivered stimulation, wherein the first group of electrodes is separated from the second group of electrodes by an electrode-free lead portion, the electrode-free lead portion having a length that is greater than each electrode pitch of the first group of electrodes and greater than each electrode pitch of the second group of electrodes.

The present technology has been developed primarily for use in/with neurostimulation of the spinal cord and will be described hereinafter mostly with reference to this application. However, it will be appreciated that the present technology is not limited to this particular field of use, and subject to modifications to lead and electrode dimensions, electrode arrangements and related matters, may be applied in other neuromodulation contexts, including sacral nerve stimulation, pudendal nerve stimulation, deep brain stimulation, stimulation of other parts of the peripheral and central nervous system, and for treatment movement disorders, Crohn's disease, rheumatoid arthritis, diabetes, Reynaud's phenomenon, incontinence/bladder disorders, faecal incontinence, non-obstructive urinary retention, constipation, chronic inflammatory conditions, migraine, stroke or depression.

### BRIEF DESCRIPTION OF THE DRAWINGS

Notwithstanding any other implementations which may fall within the scope of the present invention, one or more implementations of the technology will now be described, by way of example only, with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with target nerve fibres;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is a block diagram illustrating the data flow of a neural stimulation therapy system such as the system of FIG. 7;

FIG. 9 is an illustration of the stimulus pulses delivered by a stimulation program with four interleaved stimulation sets (stimsets);

FIG. 10 is a schematic illustrating elements and inputs of a closed-loop neural stimulation (CLNS) system with multiple stimsets;

FIG. 11 illustrates an electrode lead assembly;

FIG. 12 is a flowchart illustrating a method for selecting electrode configurations for stimulating a neural target;

FIG. 13 illustrates an electrode lead assembly comprising nine electrodes;

FIG. 14 illustrates an electrode lead assembly comprising seven electrodes;

FIG. 15 illustrates an electrode lead assembly comprising eight electrodes;

FIG. 16 illustrates an electrode lead assembly comprising slim electrodes;

FIG. 17 illustrates electrodes of an electrode arrangement configured to perform tripolar stimulation;

FIG. 18 illustrates the wavelength of an ECAP with regard to the pitch of two measurement electrodes; and

FIG. 19 illustrates an electrode lead assembly with varying electrode pitches in accordance with one implementation of the present technology.

### DETAILED DESCRIPTION OF THE PRESENT TECHNOLOGY

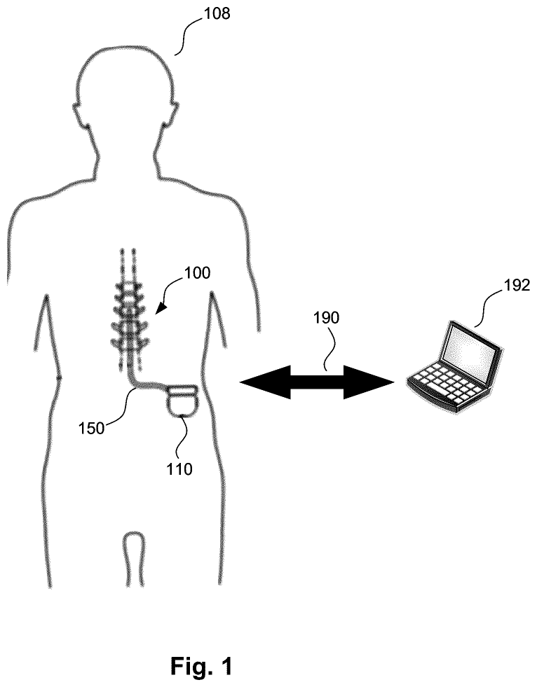

FIG. 1 schematically illustrates an implanted spinal cord stimulator 100 in a patient 108, according to one implementation of the present technology. Stimulator 100 comprises an electronics module 110 housed within a conductive case, implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode lead assembly 150 comprising an electrode arrangement implanted within the epidural space and connected to the module 110 by a suitable lead. The electrode assembly 150 may comprise one or more electrodes such as electrode pads on a paddle lead, circular (e.g., ring) electrodes surrounding the body of a percutaneous lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

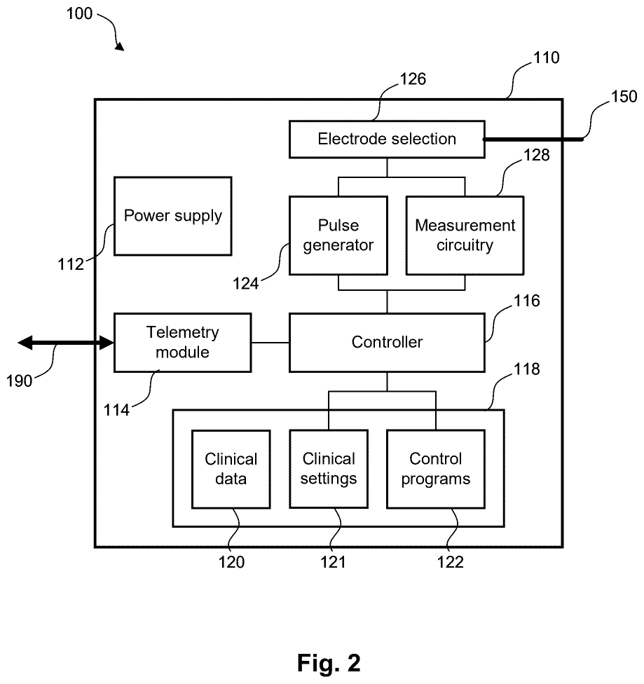

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator as part of the stimulation circuitry 124 to generate stimuli, such as in the form of electrical pulses, in accordance with the clinical settings 121. Electrode selection module 126 switches the generated pulses to the selected electrode(s) of electrode assembly 150, for delivery of the pulses to the tissue surrounding the selected electrode(s). Measurement circuitry 128, which may comprise an amplifier or an analog-to-digital converter (ADC), is configured to process signals comprising neural responses sensed by measurement electrode(s) of the electrode assembly 150 as selected by electrode selection module 126.

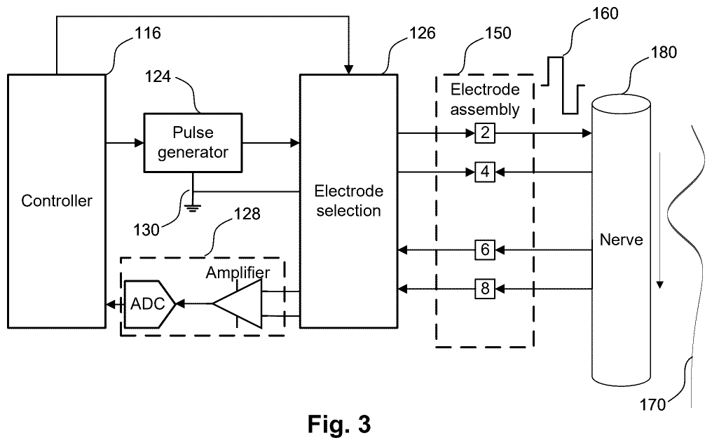

FIG. 3 is a schematic illustrating interaction of the implanted stimulator 100 with a bundle of target nerve fibres 180 in the patient 108. In the implementation illustrated in FIG. 3 the target fibres 180 may be located in the spinal cord, however in alternative implementations the stimulator 100 may be positioned adjacent any target neural tissue including a peripheral nerve, visceral nerve, sacral nerve, parasympathetic nerve or a brain structure. Electrode selection module 126 selects a stimulus electrode 2 of electrode assembly 150 through which to deliver a pulse from the pulse generator to surrounding neural tissue including target fibres 180. A pulse may comprise one or more phases, e.g. a monophasic pulse comprises one phase, and a biphasic stimulus pulse 160 comprises two phases. Electrode selection module 126 also selects a return electrode 4 of the electrode assembly 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the stimulator 100, which may therefore be configured and function as an electrode though it is not physically part of the electrode assembly 150. The set of stimulus electrodes and return electrodes is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via electrodes 2 and 4 to the target fibres 180 evokes a neural response 170 comprising an evoked compound action potential (ECAP) which will propagate along the target fibres 180 as illustrated at a rate known as the conduction velocity. The ECAP may be evoked for therapeutic purposes, which in the case of a spinal cord stimulator for chronic pain may be associated with paresthesia at a desired location. To this end, the electrodes 2 and 4 are used to deliver stimuli periodically at any therapeutically suitable stimulus frequency, for example 30 Hz, although other frequencies may be used including frequencies as high as the KHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that may be experienced by the patient as paresthesia. When a stimulus electrode configuration is found which evokes paresthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

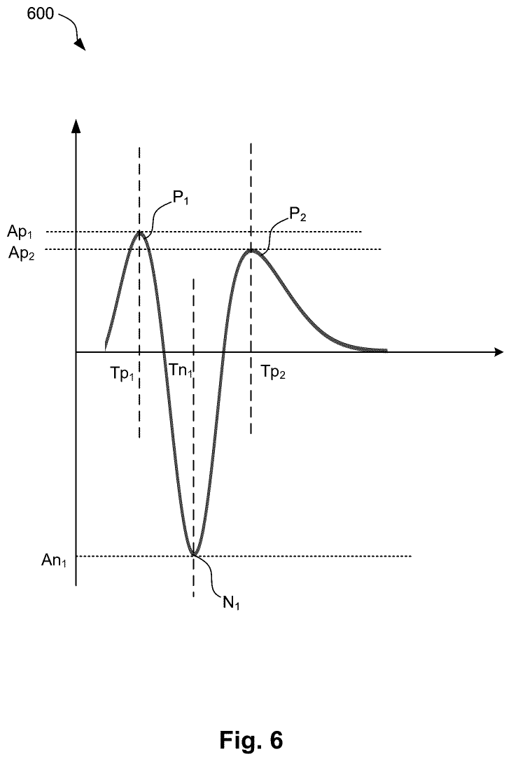

FIG. 6 illustrates the typical form of an ECAP 600 of a healthy subject, as sensed by a single measurement electrode referenced to the system ground 130 or to an indifferent electrode (a configuration referred to as single-ended ECAP measurement). The shape and duration of the single-ended ECAP 600 shown in FIG. 6 is predictable because it is a result of the ion currents produced by the ensemble of fibres depolarising and generating action potentials (A Ps) in response to stimulation. The evoked action potentials (EA Ps) generated synchronously among a large number of fibres sum to form the ECAP 600. The ECAP 600 generated from the synchronous depolarisation of a group of similar fibres comprises a positive peak P₁, then a negative peak N₁, followed by a second positive peak P₂. This shape is caused by the region of activation passing the measurement electrode as the action potentials propagate along the individual fibres. For methods and systems of selecting measurement electrodes, reference is made to International Patent Publication No. WO2023/235926, the contents of which are incorporated herein by reference.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N₁ and N₂, and one positive peak P₁. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P₁ is Ap₁ and occurs at time Tp1. The amplitude of the positive peak P₂ is Ap₂ and occurs at time Tp₂. The amplitude of the negative peak P₁ is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to measure the intensity of ECA Ps 170 propagating along target fibres 180, whether such ECA Ps are evoked by the stimulus from electrodes 2 and 4, or otherwise evoked. To this end, any electrodes of the assembly 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183, the contents of which are incorporated herein by reference.

Signals sensed by the measurement electrodes 6, 8 and processed by measurement circuitry 128 are further processed by an ECAP detector implemented within controller 116, configured by control programs 122, to obtain information regarding the effect of the applied stimulus upon the target fibres 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (uV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, target response intensity, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

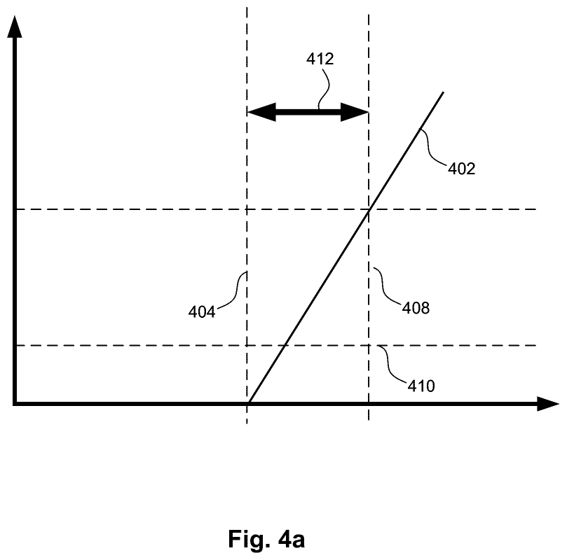

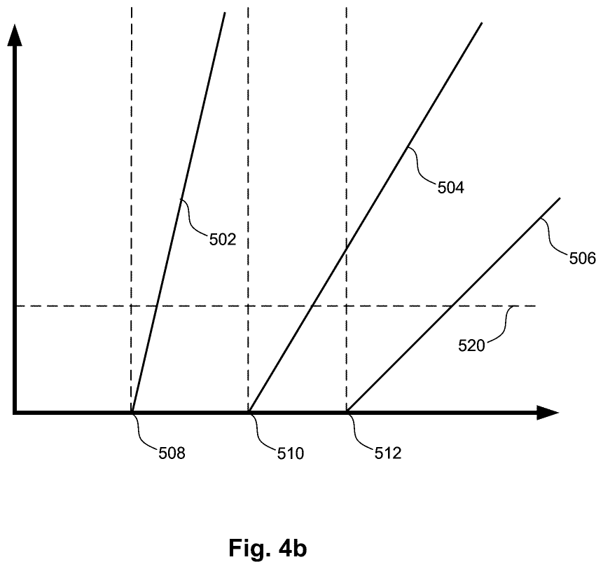

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. A bove the ECAP threshold 404, the activation plot 402 has a positive, approximately constant slope indicating a linear relationship between stimulus intensity and the ECAP amplitude. Such a relationship may be modelled as in a piecewise linear form as:

$$d = \{ \begin{matrix}S ( s - T ) , & s \geq T \\ 0 , & s < T\end{matrix} \qquad ( 1 )$$

where s is the stimulus intensity, d is the ECAP amplitude, T is the ECAP threshold and S is the slope of the activation plot (referred to herein as the patient sensitivity) above ECAP threshold T. The sensitivity S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the spinal cord. Electrode-to-cord distance is therefore loosely referred to throughout the present disclosure as posture though the two terms are not strictly synonymous. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the patient sensitivity also changes, as indicated by the varying slopes of activation plots 502, 504, and 506. In general, as the distance between the stimulus electrodes and the spinal cord increases, the ECAP threshold increases and the sensitivity decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at or near a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity to maintain a feedback variable at or near a target value is said to be operating in closed-loop mode and will also be referred to as a closed-loop neural stimulation (CLNS) device. By adjusting the applied stimulus intensity to maintain the measured ECAP amplitude or near at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at or near the target response intensity. If the target response intensity is properly chosen, the patient receives consistently comfortable and therapeutic stimulation through posture changes and other perturbations to the stimulus/response behaviour.

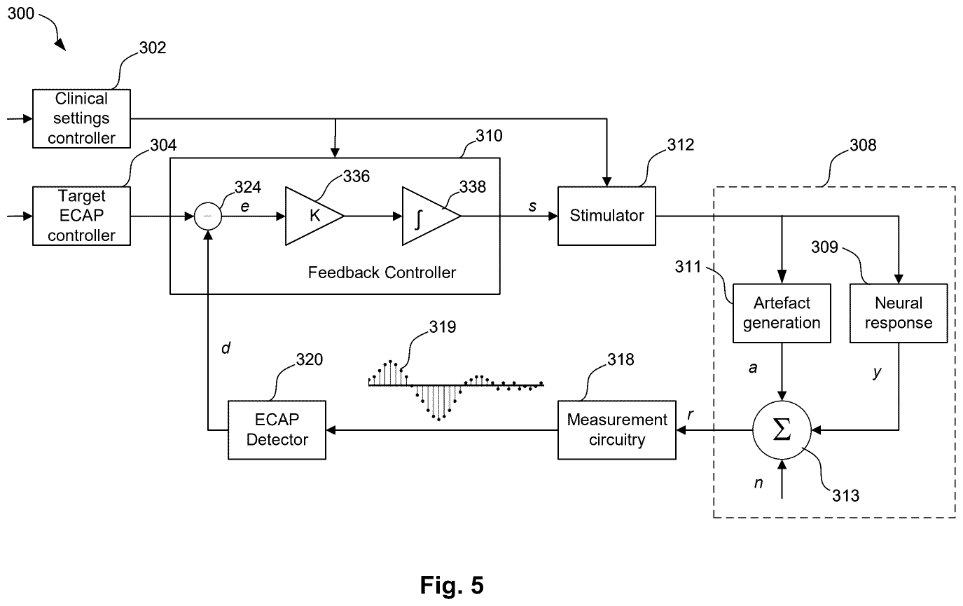

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in concert with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EM G; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode assembly or electrode assemblies. As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (potentially including evoked neural response, artefact, and measurement noise), and samples the amplified sensed signal r during a recording period to capture a “signal window” 319 comprising a predetermined number of samples of the amplified sensed signal r. The ECAP detector 320 processes the signal window 319 and outputs a measured neural response intensity d. In one implementation, the neural response intensity comprises a peak-to-peak ECAP amplitude. The measured response intensity d (an example of a feedback variable) is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int Kedt \qquad ( 1 )$$

where K is the gain of the gain element 336 (the controller gain). This relation may also be represented as

$$δ s = Ke \qquad ( 2 )$$

where ôs is an adjustment to the current stimulus intensity parameter s.

A target ECAP amplitude is input to the feedback controller 310 via the target ECAP controller 304. In one implementation, the target ECAP controller 304 provides an indication of a specific target ECAP amplitude. In another implementation, the target ECAP controller 304 provides an indication to increase or to decrease the present target ECAP amplitude. The target ECAP controller 304 may comprise an input into the CLNS system 300, via which the patient or clinician can input a target E CAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

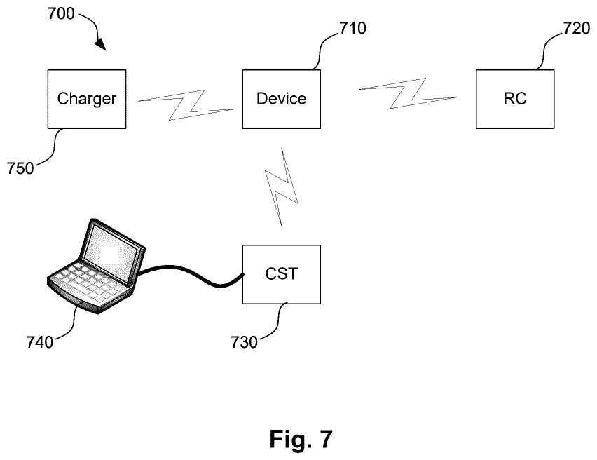

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### Data Flow

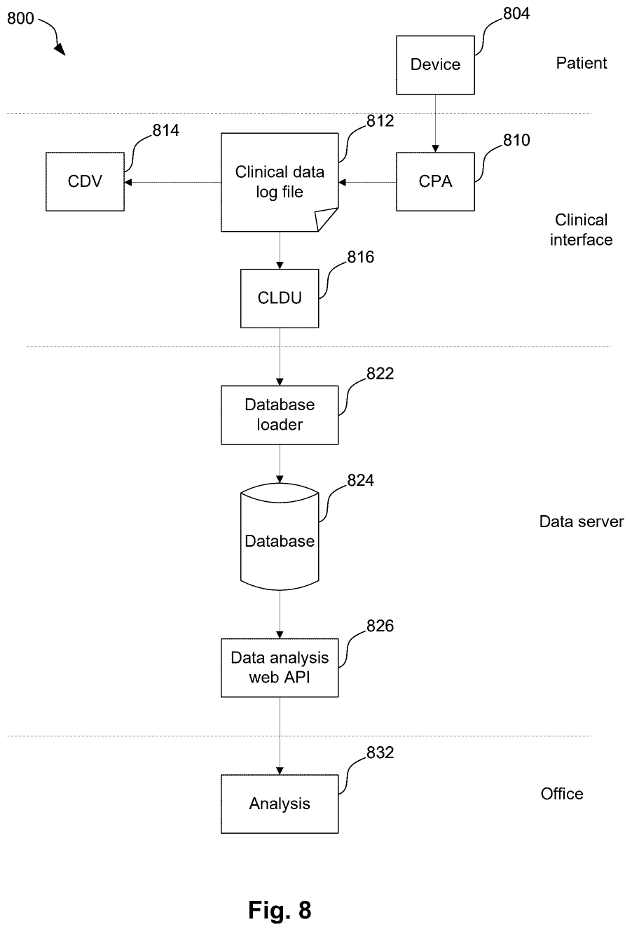

FIG. 8 is a block diagram illustrating the data flow 800 of a neural stimulation therapy system such as the system 700 of FIG. 7 according to one implementation of the present technology. Neuromodulation device 804, once implanted within a patient, applies stimuli over a potentially long period such as weeks or months and records neural responses, clinical settings, paraesthesia target level, and other operational parameters, discussed further below. Neuromodulation device 804 may comprise a Closed-Loop Neural Stimulation (CLNS) device, in that the recorded neural responses are used in a feedback arrangement to control clinical settings on a continuous or ongoing basis. To effect suitable SCS therapy, neuromodulation device 804 may deliver tens, hundreds or even thousands of stimuli per second, for many hours each day. The feedback loop may operate for most or all of this time, by obtaining sensed signals subsequent to every stimulus, or at least obtaining such sensed signals regularly. Each sensed signal generates a feedback variable such as a measure of the amplitude of the evoked neural response, which in turn results in the feedback loop changing at least one stimulus parameter for a following stimulus. Neuromodulation device 804 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 804 transmits data, e.g. via telemetry module 114, to a clinical programming application (CPA) 810 installed on a clinical interface. In one implementation, the clinical interface is the CI 740 of FIG. 7. The data can be grouped into two main sources: (1) Data collected in real-time during a programming session; (2) Data downloaded from a stimulator after a period of non-clinical use by a patient. CPA 810 collects and compiles the data into a clinical data log file 812.

All clinical data transmitted by the neuromodulation device 804 may be compressed by use of a suitable data compression technique before transmission by telemetry module 114 or before storage into the memory 118 to enable storage by neuromodulation device 804 of higher resolution data. This higher resolution allows neuromodulation device 804 to provide more data for post-analysis and more detailed data mining for events during use. Alternatively, compression enables faster transmission of standard-resolution clinical data.

The clinical data log file 812 is manipulated, analysed, and efficiently presented by a clinical data viewer (CDV) 814 for field diagnosis by a clinician, field clinical engineer (FCE) or the like. CDV 814 is a software application installed on the Clinical Interface (CI). In one implementation, CDV 814 opens one Clinical Data Log file 812 at a time. CDV 814 is intended to be used in the field to diagnose patient issues and optimise therapy for the patient. CDV 814 may be configured to provide the user or clinician with a summary of neuromodulation device usage, therapy output, and errors, in a simple single-view page immediately after log files are compiled upon device connection.

Clinical Data Uploader 816 is an application that runs in the background on the CI, that uploads files generated by the CPA 810, such as the clinical data log file 812, to a data server. Database Loader 822 is a service which runs on the data server and monitors the patient data folder for new files. When Clinical Data Log files are uploaded by Clinical Data Uploader 816, database loader 822 extracts the data from the file and loads the extracted data to Database 824.

The data server further contains a data analysis web A PI 826 which provides data for third-party analysis such as by the analysis module 832, located remotely from the data server. The ability to obtain, store, download and analyse large amounts of neuromodulation data means that the present technology can: improve patient outcomes in difficult conditions; enable faster, more cost effective and more accurate troubleshooting and patient status; and enable the gathering of statistics across patient populations for later analysis, with a view to diagnosing aetiologies and predicting patient outcomes.

### Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. There is therefore a need for a CPA to be as intuitive for non-technical users as possible while avoiding discomfort to the patient. Implementations of an Assisted Programming System (A PS) according to the present technology are generally configured to meet this need.

In some implementations, the APS comprises two elements: the Assisted Programming Module (APM), which forms part of the CPA, and the Assisted Programming Firmware (APF), which forms part of the control programs 122 executed by the controller 116 of the electronics module 110. The data obtained from the patient is analysed by the APM to determine the clinical settings for the neural stimulation therapy to be delivered by the stimulator 100. The APF is configured to complement the operation of the APM by responding to commands issued by the APM via the CST 730 to the stimulator 100 to deliver specified stimuli to the patient, and by returning, via the CST 730, measurements of neural responses to the delivered stimuli.

In other implementations, all the processing of the APS according to the present technology is done by the APF. In other words, the data obtained from the patient is not passed to the APM, but is analysed by the controller 116 of the device 710, configured by the APF, to determine the clinical settings for the neural stimulation therapy to be delivered by the stimulator 100.

In implementations of the APS in which the APM analyses the data from the patient, the A PS instructs the device 710 to capture and return signal windows to the CI 740 via the CST 730. In such implementations, the device 710 captures the signal windows using the measurement circuitry 128 and bypasses the ECAP detector 320, storing the data representing the raw signal windows temporarily in memory 118 before transmitting the data representing the captured signal windows to the A PS for analysis.

Following the programming, the APS may load the determined program onto the device 710 to govern subsequent neural stimulation therapy. In one implementation, the program comprises clinical settings 121, also referred to as therapy parameters, that are input to the neuromodulation device 710 by, or stored in, the clinical settings controller 302. The patient may subsequently control the device 710 to deliver the therapy according to the determined program using the remote controller 720 as described above. The determined program may also, or alternatively, be loaded into the CPA for validation and modification.

Multi-stimset neural stimulation

For some patients, it is beneficial for a neural stimulation therapy program to comprise multiple stimulation sets. A stimulation set (“stimset”) is a set of stimulus and return electrodes, or more precisely a stimulus electrode configuration (SEC), along with the stimulus parameters that govern the stimulation pulses delivered via that SEC.

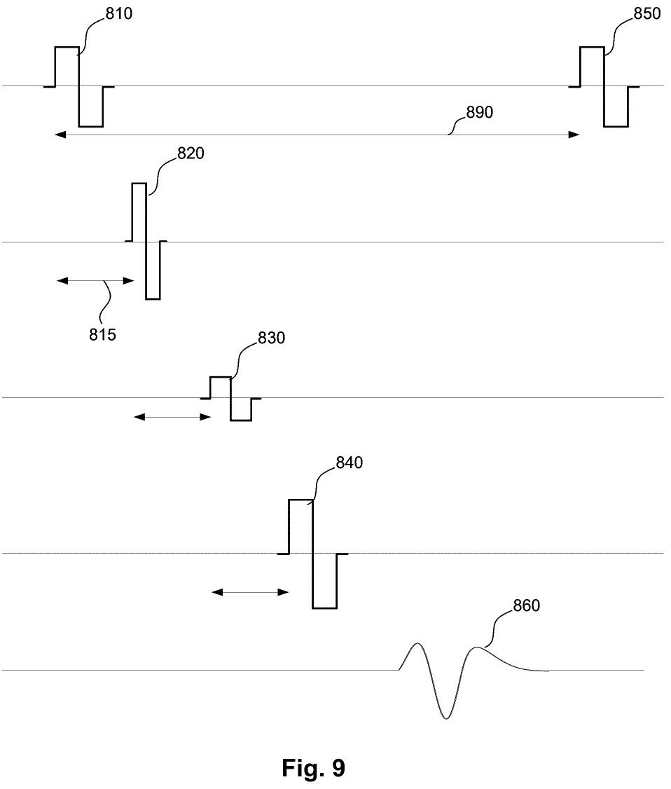

FIG. 9 is an illustration of the stimulus pulses delivered by a stimulation program with four interleaved stimsets. The stimulus pulse train delivered according to each stimset is illustrated on a separate, but vertically aligned, horizontal axis representing time. All the stimulus pulse trains are delivered at the same stimulus frequency. (It is not a requirement that all the stimulus pulse trains for the respective stimsets are delivered at the same stimulus frequency; however it is so represented in FIG. 8 for ease of illustration.) The first stimulus pulse 810, delivered according to the first stimset, is illustrated as a biphasic, anodic-first stimulus pulse, though many other stimulus pulse types are contemplated. The second, third, and fourth stimulus pulses 820, 830, and 840, delivered according to the second, third, and fourth stimsets in the program respectively, are also biphasic, anodic-first stimulus pulses with different pulse widths and different amplitudes. Each stimulus pulse is illustrated as delayed in time by a constant amount (the inter-stimulus interval, or ISI, 815) from the stimulus pulse delivered according to the preceding stimset. However, this is not to be interpreted as limiting, since the intervals between the pulses in the various stimsets may be different. Because all the stimulus pulse trains in FIG. 9 are delivered at the same stimulus frequency, the four stimulus pulses 810, 820, 830, 840 form a cycle that repeats indefinitely without any change to the relative timing of the pulses from the different stimsets. The fifth stimulus pulse 850 is a subsequent pulse in the pulse train delivered according to the first stimset and is therefore illustrated on the same time axis as the first stimulus pulse 810, and the cycle repeats thereafter. The stimulus period 890 is the period of repetition of the full cycle and is equal to the reciprocal of the stimulus frequency. In one implementation, the ISI 815 is the stimulus period divided by the number of stimsets, so that the stimuli are evenly spaced throughout the stimulus period 890.

Also illustrated is an evoked neural response in the form of an evoked compound action potential (ECAP) 860 as sensed by a predetermined measurement electrode configuration (MEC) on a common time axis with the stimulus pulses. The illustrated ECAP 860 is evoked by the fourth stimulus pulse 840. A closed-loop neural stimulation (CLNS) system programmed with multiple interleaved stimsets, as illustrated in FIG. 9, may be based on measurements of the ECAP 860. That is to say, closed-loop adjustments to the stimulus parameters of all stimsets may all be based on measurements of the ECAP 860 from a single stimset, referred to as the applied stimset. In FIG. 8, the final stimset in the cycle is the applied stimset.

If the ISI 815 is short, ECA Ps evoked by the first three stimulus pulses 810, 820, and 830 are potentially obscured by stimulus crosstalk or artefact from the stimulus pulses 820, 830, and 840. Therefore, if the ISI 815 is short, only the final stimset in the cycle may evoke a measurable ECAP. If the ISI 815 is greater than the refractory period and sufficiently long that ECA Ps evoked by the earlier stimsets are not obscured by stimulus crosstalk and artefact from the other stimulus pulses in the cycle, any of the stimsets in the cycle may evoke a measurable ECAP and may therefore be the applied stimset.

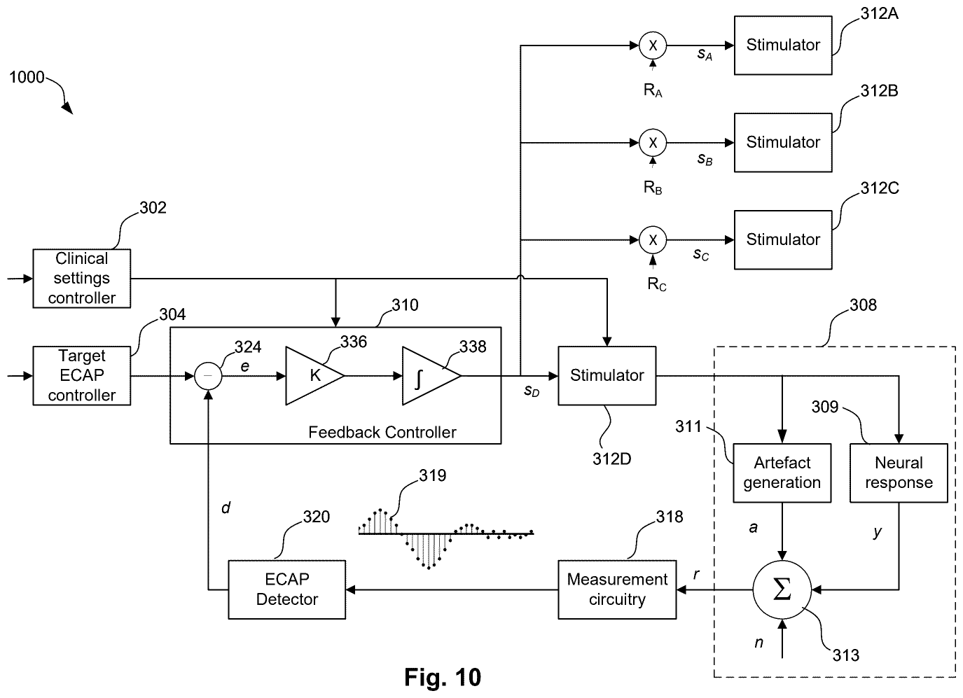

FIG. 10 is a schematic illustrating elements and inputs of a multi-stimset CL NS system 1000 with multiple stimsets. The multi-stimset CLNS system 1000 is the same as the CLNS system 300 of FIG. 5, with like numbers indicating like elements, with the addition of three further stimsets. The noise addition and artefact generation in FIG. 5 have been omitted from FIG. 10 for clarity. The four stimsets are labelled A, B, C, and D and are delivered by stimulators 312A, 312B, 312C, and 312D (the latter of which corresponds to the stimulator 312 in the CLNS system 300) according to respective stimulus intensity parameters SA, SB, Sc, and Sp, and via respective SECs. The pulses delivered by the stimulators 312A, 312B, 312C, and 312D correspond to the stimulus pulses 810, 820, 830, and 840 of FIG. 9. The neural response y may be measured from any of stimsets A, B, C, and D, delivered by the stimulator 321A, 321B, 321C, and 312D. In the implementation of FIG. 8, the stimulus intensity parameter Sp for stimset D is the largest of the four stimulus intensity parameters SA, SB, Sc and Sp and is the stimulus intensity parameter that is directly adjusted by the feedback controller 310. The stimulus intensity parameter Sp is scaled by ratios RA, RB, and Rc to obtain the stimulus intensity parameters SA, SB, and sc for stimsets A, B, and C respectively at the end of each cycle. The ratios RA, RB, and Rc which are all less than or equal to one, are fixed at the ratios of the respective stimulus intensities SA, SB, and Sc at which the respective stimsets were originally programmed, to the originally programmed stimulus intensity Sp of the applied stimset D and form part of the clinical settings 121 of the multi-stimset program. In such an implementation, the stimulus intensity parameters SA, SB, and sc always remain in fixed ratio with the stimulus intensity parameter Sp and with each other. This is referred to as ratiometric adjustment. So for example, if the originally programmed stimulus intensities were 1 mA, 2 mA, 4 mA, and 6 mA for the four stimsets A, B, C, and D respectively, the ratios RA, RB, and Rc are fixed at programming time at ⅙, ⅓, and ⅔ respectively. If during therapy the feedback controller 310 adjusts the largest stimset intensity parameter Sp to 6.6 mA, the stimulus intensity parameters SA, SB, and sc are automatically adjusted to 1.1 mA, 2.2 mA, and 4.4 mA respectively.

The clinical settings controller 302 provides to the stimulators 312A, 312B, 312C, and 312D the stimulus parameters that are not under the control of the feedback controller 310.

It may be seen from FIG. 10 that the adjustments to the stimulus intensity parameters after each stimulus cycle are all in fixed proportion. A ratiometric multi-stimset CLNS system therefore emulates a CLNS system with four separate feedback loops driven by the four stimsets, wherein each loop has the same controller gain. A ratiometric multi-stimset CLNS system is effective to maintain the responses evoked by each stimset at a constant neural response intensity on the condition that when the patient moves to a new posture, the threshold and slope of all activation plots, both for applied and non-applied stimsets, move in a proportional manner. (See FIG. 4b for examples of activation plots for a given stimset in different postures.)

Electrode lead assembly

The electrode assembly 150 comprises an arrangement of electrodes. An electrode lead assembly may comprise a plurality of electrodes arranged along an elongated body. In some implementations, each electrode comprises a ring electrode that spans 360 degrees around the long axis of the electrode lead assembly. The plurality of electrodes may be positioned in a manner to span a longitudinal distance along the lead body. Preferably, the plurality of electrodes are positioned in a manner to span a longitudinal distance along the lead body to provide flexibility in selecting electrodes for stimulation, and to increase the chance that at least some of the stimulating electrodes will be proximate to a target neural site.

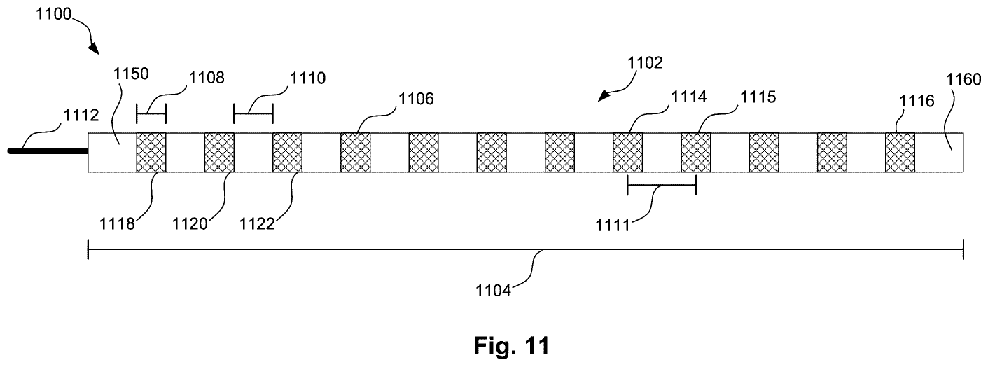

FIG. 11 illustrates an electrode lead assembly 1100, in accordance with an implementation. The electrode lead assembly comprises a body, indicated by reference span 1104. The electrode lead assembly comprises an electrode arrangement 1102 arranged on the body of the electrode lead assembly. The electrode arrangement comprises a plurality of electrodes (e.g. electrode 1106, 1114 and 1116, 1118, 1120, 1122) arranged along the body of the electrode lead assembly. In the implementation illustrated in FIG. 11, the electrode arrangement comprises 12 electrodes.

The electrode lead assembly further comprises a connector section 1112, which enables the electrode lead assembly to connect to the electronics module 110. The connector section 1112 comprises electrical connections which enable each electrode of the electrode arrangement to be electrically connected to the electronics module 110. The connector section may comprise a detachable connector, so that the electrode lead assembly may be connected to the electronics module from a disconnected state. In some implementations, the connector section is integrated with the electronics module.

The electrode lead body further comprises a start portion 1150, comprising a region from the connector section to the first electrode 1118 in the electrode arrangement. The electrode lead body further comprises an end portion 1160, comprising a region from the last electrode 1116 in the electrode arrangement to the end of the electrode lead body.

The electrode arrangement 1102 comprises twelve electrodes, wherein each electrode comprises a ring electrode that spans 360 degrees around the long axis of the electrode lead assembly. Each electrode of the electrode arrangement 1102 has a length, e.g. length 1108 of electrode 1118, which is measured along the length of the electrode lead body. In this implementation all electrodes on of the assembly 1100 have the same length 1108. Adjacent pairs of electrodes are separated by an electrode gap comprising the distance between one edge of an electrode and the opposing edge of an adjacent electrode, e.g. gap 1110 between adjacent electrodes 1120 and 1122. In this implementation adjacent pairs of electrodes on of the assembly 1100 have the same size gap 1110. Adjacent pairs of electrodes have an electrode pitch defined as the distance between the centre of an electrode and the centre of the adjacent electrode, e.g. electrode pitch 1111 of adjacent electrodes 1114 and 1115 is the distance comprising the gap between electrodes 1114 and 1115 plus the length of the electrode 1114.

In some implementations, the length of an electrode may vary from the length of another electrode in the electrode arrangement. Similarly, in some implementations, the pitch between a pair of adjacent electrodes may vary from the pitch between another pair of adjacent electrodes. Electrode selection module

A neural target, such as a spinal cord, may extend along a rostro-caudal axis of the patient. In some implementations, there is a preferred site on the neural target for stimulation in order to achieve the desired therapeutic benefit for the patient. Preferred site of stimulation may change with movement of the patient, or movement of the electrode assembly relative to the patient. To allow for shifting of the preferred stimulation site relative to the electrode assembly, either during fitting or in use, it may be desirable to select stimulus electrodes from a plurality of possible stimulus electrodes of an electrode assembly.

Preferably, the electrode lead assembly comprises electrodes which may be configured to provide stimulation, as well as electrodes which may be configured to sense a neural response resulting from the stimulation of a neural target. The sensed signal is preferably communicated to the measurement circuitry 128 of the electronics module 110 via conductors that travel along the length of the lead assembly 1100, from each electrode at a distal end region of the lead assembly 1100 to a corresponding contact at an opposing (proximal) end region of the lead assembly 1100.

In one implementation, each electrode of the electrode assembly may be electrically coupled, by the electrode selection module 126, to stimulation circuitry 124 (comprising the pulse generator), measurement circuitry 128 or neither the stimulation circuitry nor measurement circuitry. The electrode selection module 126 may comprise one or more multiplexors to couple electrodes of the electrode assembly to either the stimulation circuitry or the measurement circuitry.

For ease of reference, this disclosure may refer to an electrode that is operably connected, or coupled, to stimulation circuitry as a stimulus electrode. Similarly, this disclosure may refer to an electrode that is operably connected, or coupled, to measurement circuitry as a measurement electrode. An electrode that is operably connected to neither the stimulation circuitry nor the measurement circuitry may be referred to as an unused or passive electrode.

In some implementations, it is advantageous for an electrode to act as a stimulus electrode for one or more stimulation cycles, then for the same electrode to act as a measurement electrode for one or more further stimulation cycles. The electrode selection module 126 may couple an electrode to stimulation circuitry for one or more stimulation cycles. The electrode selection module 126 may then couple the electrode to measurement circuitry for one or more subsequent stimulation cycles. Similarly, an electrode may function as a measurement electrode for a period of time, then the same electrode may function as a stimulus electrode.

The electrode selection module 126 configures each of the electrodes of the electrode assembly to act as either: a stimulus electrode; a measurement electrode; or a passive (unused) electrode. An electrode may be considered configurable as a stimulus electrode if it may be configured to be operably connected to stimulation circuitry of an electronics module. Similarly, an electrode may be considered configurable as a measurement electrode if it may be configured to be operably connected to measurement circuitry of an electronics module.

The electrodes that the electrode selection module 126 configures to act as measurement electrodes are collectively called a measurement electrode configuration (MEC). In some implementations, a measurement electrode configuration (MEC) comprises two electrodes for differential ECAP recording, in which case the M EC may be referred to as a measurement electrode pair. The measurement electrode connected to the positive terminal of the measurement amplifier 129, via the electrode selection module 126, is referred to as the recording electrode, while the measurement electrode connected to the negative terminal of the measurement amplifier 129, via the electrode selection module 126, is referred to as the reference electrode.

The electrodes that the electrode section module 126 configures to act as stimulus electrodes are collectively called a stimulus electrode configuration (SEC). In tripolar stimulation, the SEC comprises three electrodes: a cathode electrode, configured to sink stimulus current; and two anode electrodes, configured to source return currents. The cathode electrode may be located in a position between the two anode electrodes. The three stimulus electrodes may all be located in the same row of electrodes in the rostro-caudal direction.

The electrode selection module 126, acting under control of the controller 116 of the electronics module 110, can be configured to select the M EC and SEC to achieve the desired closed loop neural stimulation of the neural target. Furthermore, in response to the measured ECAP, movement of the neural target, or movement of the electrode lead body in relation to the neural target, or a change in the desired stimulation intensity or other desired parameter, the electrode selection module 126, acting under control of the controller 116 of the electronics module 110 may adjust the MEC or the SEC to achieve the desired stimulation intensity or other desired result at the desired stimulation site.

In one implementation, the controller 116 configures the electrode selection module 126 to select the SEC to provide stimulation to a preferred site on the neural target, then the controller 116 configures the electrode selection module 126 to select the MEC in order to obtain the most accurate ECAP measurement.

In one implementation, the electrode selection module 126 selects the M EC such that there is a preferred spacing between the measurement electrodes. In one implementation, the electrode selection module 126 selects the MEC such that the positive measurement (recording) electrode sits between the SEC and the negative measurement (reference) electrode.

Method for selecting electrode configurations

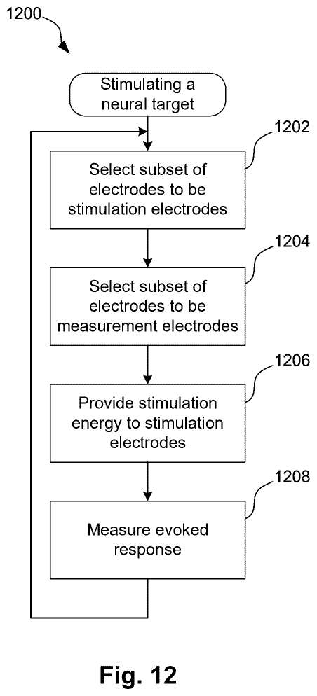

FIG. 12 is a flowchart illustrating a method for selecting electrode configurations for stimulating a neural target, according to an implementation of the present technology. In one implementation, method 1200 is performed by electronics module 110, which comprises electrode selection module 126, stimulation circuitry 124 and measurement circuitry 128, and which is electrically coupled to each of a plurality of electrodes of an electrode lead assembly. In one implementation, the electrode lead assembly comprises nine electrodes and the electrode selection module is electrically coupled to each of the nine electrodes.

In step 1202, the electrode selection module 126 selects a first subset of electrodes of the plurality of electrodes of the electrode lead assembly to function as stimulus electrodes. In one implementation, the electronics module 110 is configured to provide tripolar stimulation to a neural target in proximity to the electrode arrangement. The electrode selection module 126 selects three electrodes of the electrode arrangement as stimulus electrodes, and electrically couples the three electrodes to the stimulation circuitry 124. The stimulus electrodes may comprise two electrodes functioning as cathodes and one electrode functioning as an anode.

In step 1204, the electrode selection module 126 selects a second subset of electrodes of the plurality of electrodes of the electrode arrangement to function as measurement electrodes. The second subset of electrodes comprises two electrodes, wherein one electrode is configured to function as a recording electrode and one electrode is configured to function as a reference electrode. In one implementation, the measurement circuitry 128 is configured to measure an evoked response of the neural target by measuring a potential difference between a reference electrode and a recording electrode.

In one implementation, the electrode selection module 126 selects the second subset of electrodes based on the position of the second subset of electrodes in the electrode arrangement relative to the position of the first subset of electrodes in the electrode arrangement.

In step 1206, the electronics module 110 provides stimulation energy to the electrodes selected in step 1202. In step 1208, the electronics module 110 measures an evoked response of the neural target via the electrodes selected in step 1204, and that measured evoked response is used as input in the electronics module 110 determining the selected stimulation and measurement electrodes for the next or subsequent stimulus.

Electrode selection module configuration

In one implementation, the electrode selection module 126 performs a configuration step before step 1202. The configuration step provides the electrode selection module 126 with information about the performance of the electrodes of the electrode lead assembly, which the electrode selection module 126 may use during the selection steps 1202 and 1204.

In one implementation, the electrode selection module 126 performs the configuration step while the electronics module 110 is in-vivo, that is, while the electronics module 110 is implanted in the patient. Alternatively, or additionally, the configuration step may be performed by one or more external computing devices (e.g. device 192) and the one or more external computing devices communicate configuration information to the electrode selection module 126 via the telemetry module 114.

In one implementation, the configuration step determines information about the signal-to-artefact ratio (SAR) and signal-to-noise ratio (SNR) for various stimulus electrode configurations (SECs) and measurement electrode configurations (MECs). In one implementation, the configuration steps 1202 and 1204 comprise determining SECs and M ECs which exhibit SAR and SNR values that are above thresholds of acceptability.

It is desirable to consider the relationship between the arrangement of stimulation and measurement electrodes on an electrode lead and the artefact that may arise during use. A consideration of this relationship may inform the development of a preferred arrangement of stimulating and measurement electrodes on an electrode lead suitable for use in closed-loop spinal cord stimulation (SCS). In some implementations it is preferable to utilise tripolar stimulation, using three stimulus electrodes. Additionally, it may be preferable to use three stimulus electrodes that are proximate to each other, in accordance with a preferred stimulus electrode spacing.

Electrode artefacts

During stimulation, electric fields are produced by the stimulus electrodes. These electric fields result in field gradients that extend across the surfaces of the electrodes and may extend to the measurement electrodes. These field gradients can cause charge accumulation in the electrode-tissue interfaces, and this charge accumulation may dissipate for a period that extends well into the recording period. This charge dissipation may appear as an artefact in the measured ECAP value, and adversely affect the accuracy of the ECAP measurement. It is therefore often desirable to minimise artefact where possible.

The magnitude and impact of an artefact in the measured ECAP value can be affected by the configuration of the electrodes in the electrode arrangement. For example, the relative positioning of the stimulus and measurement electrodes can affect the artefact, as can the size and shape of the electrodes.

It is preferable to have a high signal to artefact ratio with regard to the measured response of the tissue, e.g. the measured ECAP response. In some implementations, it is preferable for the artefact component of the measured ECAP response to be less than 10 μV in response to a stimulation of 1 μC (200 μs, 5 mA) with attenuating factor @=1 (as defined below).

The signal to artefact ratio (SAR) may be described by Equation (4).

$$SAR = \frac{s}{A} \qquad ( 4 )$$

Where S describes the ECAP component of the signal recorded by a measurement electrode pair, and A is the measured artefact in the configuration.

The size of the recorded ECAP varies with the distance between the measurement electrodes as the ECAP voltage along the cord has an approximate sinusoidal shape. M oving the measurement electrodes closer, or further away may effect the magnitude of the recorded ECAP signal.

A theoretical value for the expected signal value for a measurement electrode pair can be derived using a reference ECAP value. This theoretical value captures the effect of distance between planar/cylindrical electrodes on ECAP value. Using the lowest clinically useful values ensures the results are relevant in situations where low ECAP signal is expected. This value does not capture how the signal is attenuated by the spatial arrangement of the measurement electrode pair relative to the SEC.

A simple model that captures the effect of distance between measurement electrodes is described by Equation (5).

$$S = ω R \qquad ( 5 )$$

where S described the E CAP component of the signal recorded by a measurement electrode pair, R is the reference ECAP value and @ is an attenuating factor described by Equation (6).

$$ω = sin ( \frac{π d}{18 mm} ) \qquad ( 6 )$$

where d describes the distance between the two measurement electrodes.

Reduced number of electrodes

An electrode lead assembly that provides a multitude of stimulation configuration options, may provide for the selection of an electrode in a preferred location relative to a neural target to affect a desirable stimulation of that neural target. It is desirable to balance the ability of an electrode lead assembly to provide a multitude of stimulation configuration options, with other factors, such as manufacturing cost, artefact issues and operational management issues.

Advantageously, an electrode lead assembly with a smaller number of electrodes may incur reduced manufacturing costs in comparison to an electrode lead assembly comprising a larger number of electrodes. An electrode lead assembly with fewer electrodes (and consequently fewer contacts at a proximal end) may have a reduced length and be less susceptible to buckling or bending of the proximal end of the lead assembly that may occur during insertion into a pulse generator (e.g. see 110 of FIG. 1). Such buckling or bending can hinder insertion, and is in part due to friction forces that resist insertion of the proximal end where it is inserted into tightly fitting cavities in the pulse generator, the forces increasing depending on the length of proximal end to be inserted. A shorter proximal end, with fewer contacts connected to corresponding electrodes at a distal end of the lead assembly, is therefore desirable. Additionally, the risk of an electrode-specific manufacturing error or an electrode-specific operational error occurring in the electrode lead assembly may be reduced with a reduction in the number of electrodes in the electrode lead assembly.

### Electrode Free Portions

In some implementations, it is desirable to include electrode free portions in the electrode lead assembly. An electrode free portion is a portion of the lead assembly that does not comprise an electrode, though it can comprise conductors within the body of the lead assembly surrounded by a non-conductive material.

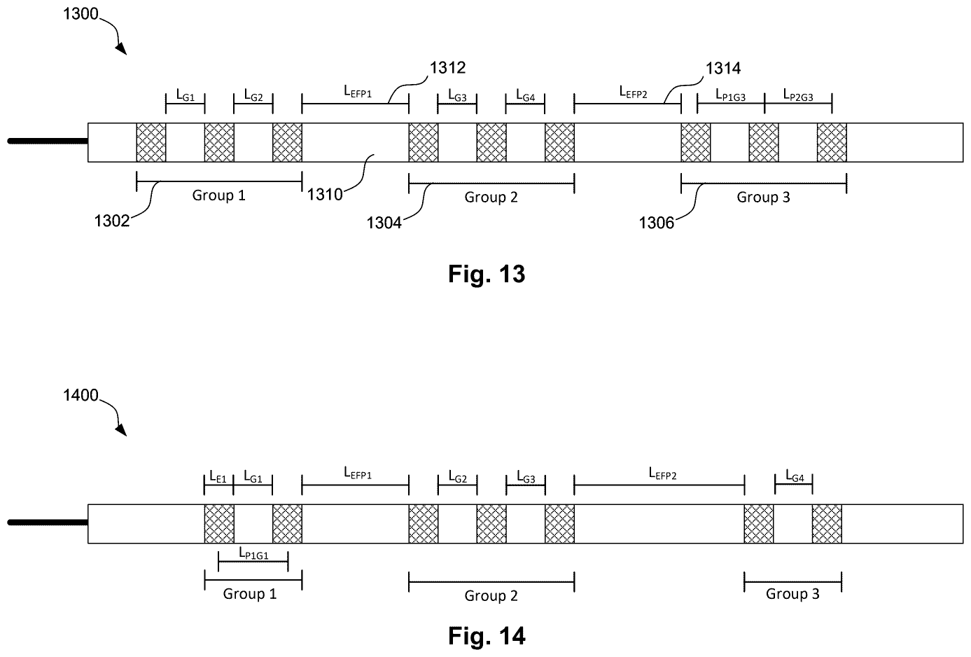

FIG. 13 illustrates an electrode lead assembly 1300 comprising nine electrodes, in accordance with a preferred implementation of the present technology. The nine electrodes are arranged into three groups of three electrodes, specifically Group 1 electrodes 1302, Group 2 electrodes 1304 and Group 3 electrodes 1306. A group of electrodes comprises two outer electrodes, and one or more middle electrodes.

A comparison of the electrode lead assembly 1100 with the electrode lead assembly 1300 shows that electrodes 1106, 1114 and 1116 are omitted from electrode lead assembly 1300. Instead, the electrode lead assembly 1300 comprises a first electrode free portion (EFP) 1310 (also referenced by distance 1312) that separates the Group 1 electrodes and the Group 2 electrodes, and a second electrode free portion 1314 that separates the Group 2 electrodes and the Group 3 electrodes. The length of the electrode free portion between the Group 1 electrodes and the Group 2 electrodes is indicated by length L_{EFP1}. The length of the electrode free portion between the Group 2 electrodes and the Group 3 electrodes is indicated by length LEFP2.

Adjacent pairs of electrodes in Group 1 have a respective electrode pitch defined as the distance between the centre of an electrode and the centre of the adjacent electrode. Similarly, adjacent pairs of electrodes in Groups 2 and 3 have a respective electrode pitch. For illustrative convenience, only the electrode pitches, L_{P1G3} L_{P2G3}, between the two pairs of adjacent electrodes in Group 3 are marked in FIG. 13.

Each adjacent pair of electrodes in Group 1 are separated, respectively, by inter-electrode gap L_{G1} and L_{G2}. Similarly, each adjacent pair of electrodes in Group 2 are separated, respectively, by inter-electrode gap L_{G3} and L_{G4}. In this implementation, inter-electrode gaps L_{G1}, L_{G2}, L_{G3} and L_{G4} are equal in length; however, in other implementations inter-electrode gaps may vary along the electrode lead.

As used herein, a group of electrodes comprises one or more electrodes that are bounded by any two of: the start portion of the electrode lead; the end portion of the electrode lead; and an electrode free portion. As used herein, an electrode pitch of a group of electrodes comprises the longest of the electrode pitches between each adjacent pair of electrodes in the group of electrodes.

Preferably the electrode free portion, between a first group of electrodes and a second group of electrodes, is longer than the electrode pitches between adjacent electrodes in both electrode groups. Alternatively, the electrode free portion may be longer than the inter-electrode gap. As illustrated in FIG. 13, the electrode free portion L_{EFP1} is longer than the inter-electrode gaps L_{G1} and L_{G2} between adjacent Group 1 electrodes. Additionally, the electrode free portion L_{EFP1} iS longer than the inter-electrode gaps L_{G3} and L_{G4} between adjacent Group 2 electrodes.

Advantageously, the longer electrode free portion, relative to the electrode pitch between adjacent electrodes in the neighbouring groups of electrodes, acts to reduce the artefact generated as a result of stimulation at a given intensity via a SEC selected from electrodes in one group and incident on the measurement electrodes selected from electrodes in the neighbouring group.

In some implementations, the longer electrode free portion, relative to the electrode pitch between adjacent electrodes in the neighbouring groups of electrodes, provides a flexible region, allowing the electrode lead to bend to more effectively position the electrodes in proximity to the neural target.

In some implementations, the longer electrode free portion, relative to the electrode pitch between adjacent electrodes in the neighbouring groups of electrodes, allows for the provision of a higher intensity of stimulation from a SEC in one electrode group without causing the resulting artefact, sensed by measurement electrodes selected in the neighbouring electrode group, to exceed an acceptable level.

Simulation configuration options

Electrodes in each group of electrodes (1302, 1304 and 1306) may be configured, by the electrode selection module 126, to function variously as either a stimulation or a measurement electrode. The 3×3 electrode arrangement, illustrated in FIG. 13, is particularly advantageous as it provides several stimulation sites for tripolar stimulation (e.g. one site for each group of electrodes).

A 3×3 electrode arrangement provides for at least six different combinations of stimulus and measurement electrode configurations (i) Group 1 electrodes configured for stimulation and Group 2 electrodes configured for measurement; (ii) Group 1 electrodes configured for stimulation and Group 3 electrodes configured for measurement; (iii) Group 2 electrodes configured for stimulation and Group 1 electrodes configured for measurement; (iv) Group 2 electrodes configured for stimulation and Group 3 electrodes configured for measurement; (v) Group 3 electrodes configured for stimulation and Group 1 electrodes configured for measurement; and (vi) Group 3 electrodes configured for stimulation and Group 2 electrodes configured for measurement. Also, combinations of the above may be possible, such as a configuration being a combination of (iii) and (iv), where Group 2 electrodes are configured for stimulation, and Groups 1 and 3 are configured for measurement.

In some implementations, the electrodes from multiple groups of electrodes may be configured to be stimulus electrodes, to increase the therapeutic range of applications. For example, the electrodes of Group 1 and Group 2 may be electrically connected to the stimulation circuitry 124 to act as stimulus electrodes, whilst the electrodes of Group 3 are connected to the measurement circuitry 128 to act as measurement electrodes.

Similarly, in some implementations, the electrodes from multiple groups of electrodes may be configured to be measurement electrodes, to increase the breadth or accuracy of ECAP response measurements.

The pitch and spacing of electrodes, including the electrode free portion, is preferably chosen to balance the trade-offs between managing artefact arising from stimulus electrodes incident on the measurement electrodes, and measuring the peak-to-peak differential ECAP measurement sensed by two measurement electrodes, while providing a broad range of selectivity for stimulus locations.

Alternative electrode arrangements

The preferred electrode configuration may depend on the size, position and nature of the neural target. Similarly, the preferred electrode configuration may depend on manufacturing or operational factors.

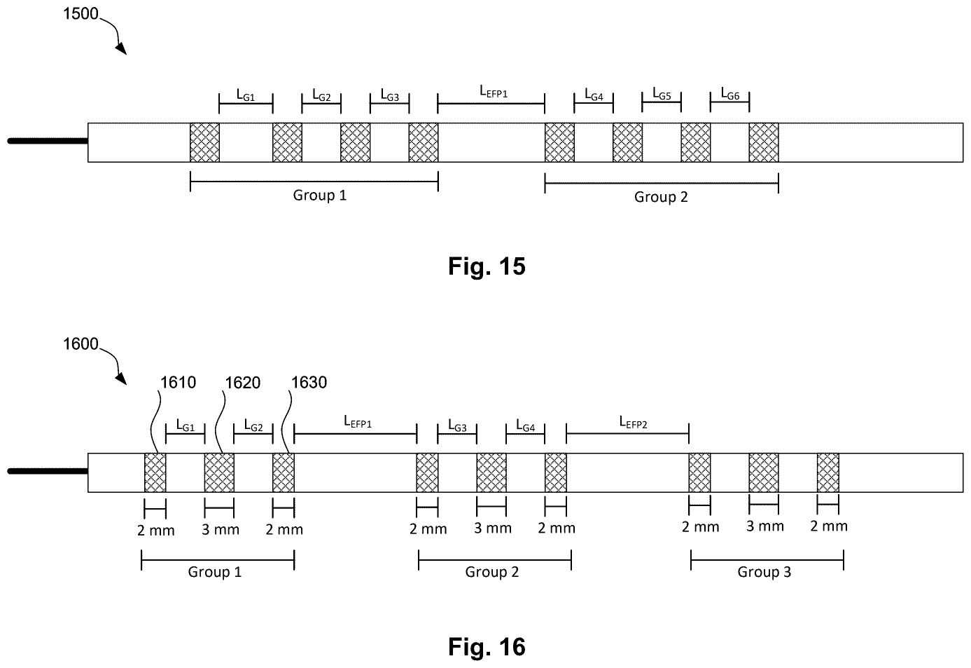

FIGS. 14 and 15 illustrate alternative preferred electrode configurations. In particular, FIG. 14 illustrates another electrode lead assembly 1400, in accordance with a preferred implementation of the present technology. Electrode lead assembly 1400 comprises seven electrodes, grouped into three groups of electrodes, wherein each group of electrodes are separated by an electrode free portion. Groups 1 and 3 each comprises two electrodes, and Group 2 comprises three electrodes.

For electrode lead assembly 1400, the electrode pitch for Group 1, LPIGI is less than the electrode free portion L_{EFP1}. The electrode pitch for Group 1, LPiGiis approximately 2 times the electrode length, LE1.

Additionally, for electrode lead assembly 1400, the electrode free portion L_{EFP1} is longer than the inter-electrode gap L_{G1} of the Group 1 electrodes, and the electrode free portion L_{EFP1} is also longer than the inter-electrode gaps (L G2 and L G3) of the Group 2 electrodes.

Similarly, the electrode free portion LEFP2 is longer than the inter-electrode gaps (L_{G2} and L_{G3}) of the Group 2 electrodes, and the electrode free portion LEFP2 is also longer than the inter-electrode gap L_{G4} of the Group 3 electrodes. L_{EFP1} and LEFP2 are each longer than the pitch of the Group 1 electrodes (and the pitch of the Group 2 and Group 3 electrodes, which have the same pitch as the Group 1 electrodes, LP1G1). The design of the lead assembly of FIG. 14 provides a spacing between the Group 2 and Group 3 electrodes (the electrode free portion L EFP2) that reduces the stimulation induced artefact caused by the Group 2 electrodes operating as stimulation electrodes and sensed by the Group 3 electrodes operating as measurement electrodes.

The length of electrode free portions may vary along an electrode arrangement, to better suit manufacturing or operational constraints, or to position the electrodes in closer proximity to the neural target. For the implementation illustrated in FIG. 14, the electrode free portion LEFP2 IS longer than the electrode free portion L_{EFP1}.

FIG. 15 illustrates another electrode lead assembly 1500, in accordance with a preferred implementation of the present technology. Electrode lead assembly 1500 comprises eight electrodes, grouped into two groups of four electrodes, wherein the groups of electrodes are separated by an electrode free portion.

Notably, for electrode lead assembly 1500, the electrode free portion L_{EFP1} is longer than the pitch between the most proximal two electrodes in Group 1, and the pitch between the remaining two pairs of electrodes in Group 1. Similarly, electrode free portion L_{EFP1} is longer than the pitch between each adjacent pair of electrodes in Group 2. Electrode free portion L_{EFP1} is also longer than inter-electrode gaps (L_{G1}, L_{G2}, and L_{G3}) of the Group 1 electrodes, and the electrode free portion L_{EFP1} is also longer than the inter-electrode gaps (L_{G4}, LG5, and LG6) of the Group 2 electrodes.

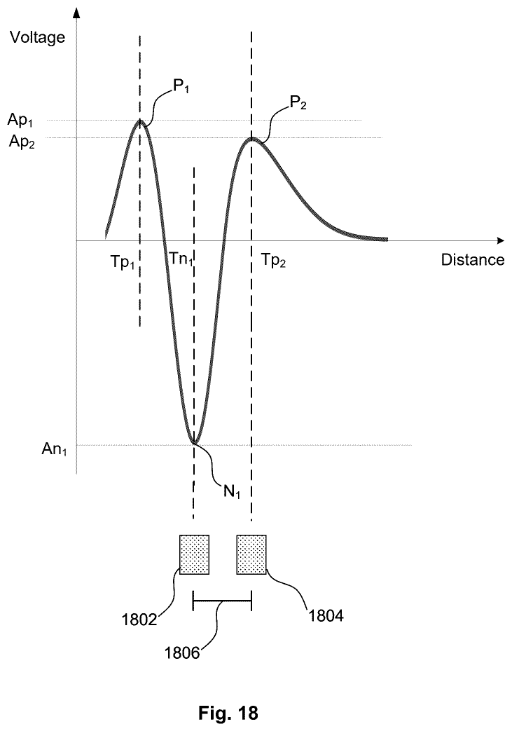

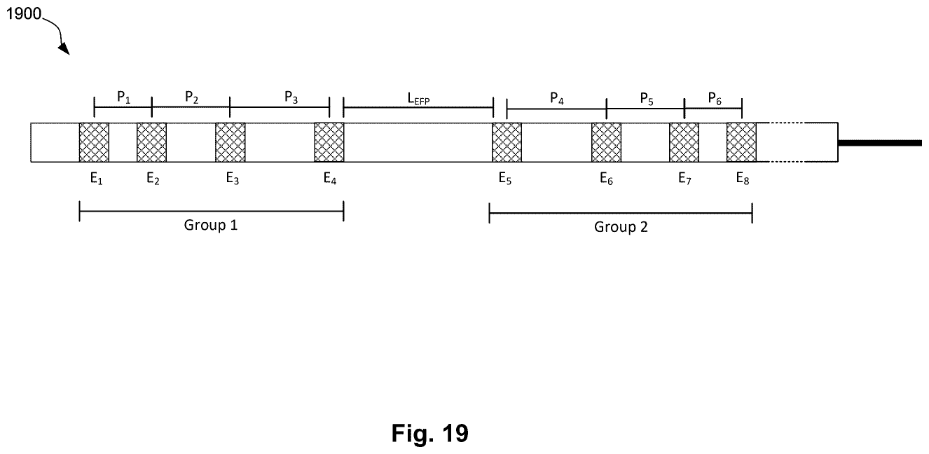

Inter-electrode gaps and/or pitches between adjacent pairs of electrodes, may vary along an electrode arrangement, to better suit manufacturing or operational constraints, or to position the electrodes in closer proximity to the neural target. An implementation with linearly varying electrode pitches is illustrated in FIG. 19. For the implementation illustrated in FIG. 15, the inter-electrode gap L_{G1} is longer than each of the inter-electrode gaps L_{G2}, L_{G3}, L_{G4}, LG5, and LG6. Similarly, the pitch between the two most proximal electrodes of Group 1 is greater than the pitches between other pairs of adjacent electrodes in Group 1 and Group 2. Variations in pitch between pairs of electrodes permits electrode spacings to be chosen to be particularly suitable allowing the measurement circuitry to capture the entire, or substantially the entire, Ni peak to P₂ peak differential (see FIG. 18 herein and corresponding text).

Preferably, adjacent electrodes of a group of electrodes in an electrode arrangement have an electrode pitch of between about 3 millimetres and about 7 millimetres. In some implementations, the electrode pitch may be greater than 7 millimetres.

Reduced length

To ameliorate the effect of electrode artefacts, electrodes may be configured to be shorter in the rostro-caudal dimension (e.g. along the length of the electrode lead body). Such electrodes may be referred to as slim electrodes. In some implementations, it is preferable that the two outer electrodes in a group of electrodes comprise slim electrodes.

FIG. 16 illustrates an electrode lead assembly 1600, in accordance with a preferred implementation of the present technology. The electrode arrangement of the electrode lead assembly 1600 comprises a 3×3 electrode arrangement, in which nine electrodes are arranged in three groups of three electrodes. The electrode lead assembly 1600, like the electrode lead assembly 1300, provides several stimulation sites for tripolar stimulation (e.g. one site for each group of electrodes).

The two outer electrodes in each group of three electrodes are slim electrodes, having a reduced electrode length, as measured along the length of the electrode lead body. In particular, of the three electrodes in Group 1, middle electrode 1620 has an electrode length of about 3 millimetres, and the two outer electrodes (1610 and 1630) have a length of about 2 millimetres.

In implementations, an electrode arrangement may comprise one or more slim electrodes in a group of any number of electrodes. For example, a group of two electrodes may comprise two slim electrodes; a group of four electrodes may comprise two slim electrodes located on the outer positions of the group and two wider electrodes located on the inner positions of the group.

Preferably, a slim electrode has a length of 1.5 millimetres (or about 1.5 millimeters) to 2 millimetres (or about 2 millimetres) to minimise artefact.

Tripolar stimulus electrode configuration

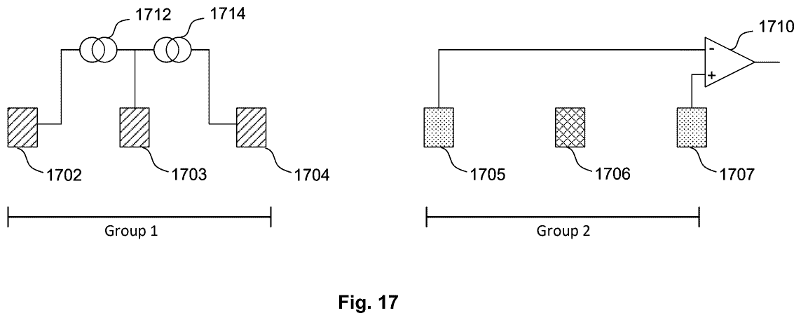

FIG. 17 illustrates a plurality of electrodes of an electrode arrangement, configured to perform tripolar stimulation, in accordance with a preferred implementation of the present technology. FIG. 17 illustrates six electrodes, 1702 to 1707, of an electrode arrangement of an electrode lead assembly, according to one implementation of the present technology. The electrode assembly is configured to be implanted in the patient such that the row of six electrodes 1702 to 1707 are substantially in alignment along the rostro-caudal axis of the patient. Electrodes 1702, 1703 and 1704 are coupled to stimulation circuitry 124, via the electrode selection module 126, and are therefore configured to function as stimulus electrodes (as indicated by the diagonal fill pattern). The stimulus electrodes, 1702, 1703 and 1704, receive stimulation energy from the stimulation circuitry 124 via current sources 1712 and 1714.

Electrodes 1705 and 1707 are coupled to measurement circuitry 128 via the electrode selection module 126 and are therefore configured to function as measurement electrodes (as indicated by the dot fill pattern). Measurement electrodes 1705 and 1707 are configured to enable measurement of an ECAP response of the neural tissue. The ECAP response of the tissue is differentially measured by determining a voltage across electrodes 1705 and 1707, as amplified by amplifier 1710.

In this example of tripolar stimulation, electrode 1706 is a passive electrode (as indicated by the cross-hash fill pattern).

Pitch of measurement electrodes

FIG. 18 illustrates the wavelength of an ECAP (as described in relation to FIG. 6) with regard to the pitch of two measurement electrodes, according to one implementation of the present technology. The measurement electrodes comprise a recording electrode 1802 and a reference electrode 1804. The recording electrode 1802 and the reference electrode 1804 may or may not be adjacent electrodes in the electrode arrangement. One or more other electrodes may be positioned between the recording electrode 1802 and the reference electrode 1804. One or more electrode free portions may be positioned between the recording electrode 1802 and the reference electrode 1804.

A preferred pitch 1806 of the measurement electrodes (i.e. distance between the centre of reference electrode 1804 and the centre of recording electrode 1802) allows the measurement circuitry to capture the entire, or substantially the entire, N₁ peak to P₂ peak differential.

As mentioned above, differential measurement between electrodes in the arrangement is preferable to reduce artefact that arises from measurement with a single measurement electrode using the case as a reference, or common-mode noise arising on surrounding tissue from single-ended ECAP measurement using the system ground 130 as a reference.

In some implementations, it is preferable that the pitch 1806, i.e. the distance between the centre of a first measurement (i.e. recording) electrode 1802 and the centre of a second measurement (i.e. reference) electrode 1804, is 2 times, or about 2 times, the pitch of adjacent stimulus electrodes. In some implementations, it is preferable that the pitch, i.e. the distance between the centre of a recording electrode and the centre of a reference electrode, is about, or slightly greater than, 1.5 to 2 times the pitch of adjacent stimulus electrodes. It is reiterated that, depending on the configuration, individual electrodes can be configured as nominal stimulus or nominal measurement electrodes.

In some implementations, particularly where slim electrodes are employed, an inter-electrode distance within a range of about 2 and about 3.66 times electrode length is preferred. Electrode lengths of about or less than about 3 millimetres, and particularly of about 2 millimetres are disclosed. Such arrangements for electrode spacing and electrode lengths are desirable to reduce artefact, and are set out in International Patent Publication No. WO2020/082126, the contents of which is incorporated herein by reference. In one implementation, artefact is able to be reduced where the lead assembly comprises electrodes having length of 2 millimetres, and ratio of inter-electrode distance to electrode length of about 2.5.

Varying pitch, including continuously varying pitch within an electrode group

With reference to FIG. 19, an implementation of the present technology is illustrated as electrode lead assembly 1900 comprising an electrode lead body and a plurality of electrodes arranged along a length of the electrode lead body. The plurality of electrodes comprise a first group of electrodes (Group 1) and a second group of electrodes (Group 2), each group of electrodes comprising four electrodes, wherein adjacent pairs of electrodes in each group of electrodes have a respective electrode pitch. Each electrode is configurable as a stimulus electrode for delivering stimulation to a neural target, or as a measurement electrode for measuring a response evoked by the delivered stimulation, to provide a broad range of therapeutic coverage.

The Group 1 electrodes are located proximally of the Group 2 electrodes along the lead assembly.

The plurality of electrodes comprise two middle electrodes, E₄ and E₅. The electrode pitch for adjacent pairs of electrodes increases for adjacent pairs closer to the one or more middle electrodes. More specifically, the pitch between the two most proximal (E₁ and E₂) electrodes and the two most distal electrodes (E₇ and E₈), P₁ and P₆, respectively, is smaller than the pitch between the next most proximal pair of electrodes, P₂, and the pitch between the next most distal pair of electrodes, P₅, which in turn are smaller than the pitch between the still next most proximal pair of electrodes, P₃, and the pitch between the still next most distal pair of electrodes, P₄. In some electrode arrangements, the profile of pitch variation is symmetrical about the middle electrodes of the lead body (e.g., the difference between P₁ and P₂ is equal to the difference between P₆ and P₅, and the difference between P₂ and P₃ is equal to the difference between P₅ and P₄). In some electrode arrangements, the profile of pitch variation is asymmetrical about the middle electrodes of the lead body (e.g., the difference between P₁ and P₂ is not equal to the difference between P₆ and P₅, or the difference between P₂ and P₃ is not equal to the difference between P₅ and P₄).

The pitch of adjacent electrodes in Groups 1 and 2 varies in a linear profile, increasing by 3 millimetres for each adjacent pair that is closer to the middle electrodes. In Group 1, P₁ is 6 millimetres, P₂ is 9 millimetres and P₃ is 12 millimetres. The electrode free portion LEFP is 15 millimetres, though it may be less than P₃ or 12 millimetres in other electrode arrangements. Similarly, within Group 2, P₆ is 6 millimetres, Ps is 9 millimetres and P₄ is 12 millimetres. In alternative electrode arrangements, the pitch may vary in a Gaussian profile, increasing non-linearly from most proximal to the middle electrodes and decreasing symmetrically to most distal.

In alternative electrode arrangements, there may be more than two groups of electrodes arranged in a similar manner, having increasing or decreasing pitches between adjacent electrodes.

This electrode arrangement provides means to achieve a pitch of a pair of measurement electrodes of 1.5 to 2 times the pitch of a pair of adjacent stimulus electrodes, due to the varying pitches of adjacent pairs of electrodes. For example, the electrode pairs at the most distal and proximal ends of the electrode arrangement can be configured as stimulus electrodes (i.e. E₁ and E₂, E₇ and E₈), and pairs of electrodes closer to the middle electrodes (i.e. E₃ and E₄, or Es and E₆) can be configured as measurement electrodes.

Notably, depending on the total number of electrodes in the electrode arrangement, where a symmetrically varying pitch is implemented, greater selectivity of activated fibres can be achieved at the most distal and proximal regions of the electrode arrangement. This can be particularly helpful from a programming perspective, as greater selectivity of activated fibres (and consequent greater control of stimulation location) is desirable at extreme ends of the electrode arrangement.

Paddle lead

Electrode arrangements disclosed herein, may be implemented in an electrode lead assembly comprising a paddle lead. In a paddle lead implementation, the electrodes may comprise rectangular electrodes, for example stamped electrodes with a raised patient contacting surface.

For example, a paddle lead may comprise two rows of rectangular electrodes, each row containing two or three groups of electrodes in the arrangement as set out in FIG. 13 to 17, or 19. Rectangular electrodes for a paddle lead may be oriented so their longitudinal axis extends rostro-caudally. For example, a paddle lead implementation may comprise 16 electrodes, including a first row having eight electrodes, namely a first group of four electrodes that is proximal to a second group of electrodes also having four electrodes, the first and second groups of electrodes being separated by a first electrode free portion that is greater than the electrode pitch of the first and second group of electrodes. Such a paddle lead may further comprise a second row of electrodes, spaced laterally on the lead beside the first row, the second row also having eight electrodes, namely a third group of four electrodes that is proximal to a fourth group of electrodes also having four electrodes, the third and fourth groups of electrodes being separated by a second electrode free portion that is greater than the electrode pitch of the third and fourth group of electrodes.

It will be appreciated that the present application seeks to provide an electrode lead assembly, and methods of measuring neural responses using an electrode lead assembly, which balance two or more factors, including:
the ability to select electrodes to be stimulus electrodes from electrodes positioned at a plurality of stimulation sites along an electrode lead assembly;
the ability to deliver multi-dermatomal stimulation from spaced stimulation electrodes;
flexibility in selecting measurement electrode location relative to the stimulation site, where there are further competing factors of (i) where measurement and stimulation electrodes are close to each other, artefact increases; and (ii) where measurement and stimulation electrodes are too far apart, the neural response (e.g. ECAP) attenuates (e.g. due to dispersion and synapsing)
providing an optimal distance between measurement electrodes (i.e. recording and reference electrodes) to maximise capturing the peak-to-peak ECAP recording, yet also maximise the signal-to-artefact ratio or the signal-to-noise ratio;
the cost of electrodes or other materials required for longer electrode lead assemblies;
buckling or bending of the proximal portion of the lead assembly during insertion into an implantable pulse generator;
electrode length to pitch ratio to maximise the signal-to-noise ratio or the signal-to-artefact ratio.

It will be appreciated that, particularly in the context of closed loop stimulation, finding a preferred balance of the above factors can provide substantial benefits to delivering cost-effective and efficacious therapeutic solutions.

### Implementations

Reference throughout the present disclosure to “one implementation” or “an implementation” means that a particular feature, structure or characteristic described in connection with the implementation is included in at least one implementation of the present technology. Thus, appearances of the phrases “in one implementation” or “in an implementation” in various places throughout the present disclosure are not necessarily all referring to the same implementation, but may refer to different implementations. Furthermore, the particular features, structures or characteristics may be combined in any suitable manner, as would be apparent to one of ordinary skill in the art from this disclosure, in one or more implementations.

Similarly, it should be appreciated that in the above description of example implementations of the present technology, various features are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects may lie in less than all features of a single foregoing disclosed implementation. Thus, the claims following the Detailed Description of the Present Technology are hereby expressly incorporated into this Detailed Description of the Present Technology, with each claim standing on its own as a separate implementation of this present technology.

Furthermore, while some implementations described herein include some but not other features included in other implementations, combinations of features of different implementations are meant to be within the scope of the present technology, and form different implementations, as would be understood by those in the art. For example, in the following claims, any of the claimed implementations can be used in any combination.

As used herein in the specification and claims, including as used in the examples and unless otherwise expressly specified, all numbers may be read as if prefaced by the word “about” or “approximately,” even if the term does not expressly appear. The phrase “about” or ‘approximately” may be used when describing magnitude or position to indicate that the value or position described is within a reasonable expected range of values or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that each unit between two particular units are also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that implementations of the present technology may be practiced without these specific details. In other instances, well-known methods, structures and techniques have not been shown in detail in order not to obscure an understanding of the present technology.

### Terminology

Throughout this specification, the terms “a” and “an” mean “one or more”, unless expressly specified otherwise.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” or “one or more of’ a list of options is to be understood to mean that the element may be any one of the listed options, or may be any combination of two or more of the listed options.

In this specification the word “or” is to be read inclusively rather than exclusively, except where otherwise indicated.

Neither the title nor any abstract of the present application should be taken as limiting in any way the scope of the claimed invention.

The referenced Figures serve to illustrate embodiments of the described technology and may not be drawn to scale.

Where the preamble of a claim recites a purpose, benefit or possible use of the claimed invention, it does not necessarily limit the claimed invention to having only that purpose, benefit or possible use.

In the present specification, terms such as “part”, “component”, “means”, “section”, or “segment” may refer to singular or plural items and are terms intended to refer to a set of properties, functions, or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limited to direct connections only. Thus, the scope of the expression “an item A connected to an item B” should not be limited to items or systems wherein an output of item A is directly connected to an input of item B. It means that there exists a path between an output of A and an input of B which may be a path including other items or means. “Connected”, or a similar term, may mean that two or more elements are either in direct physical or causal contact, or that two or more elements are not in direct contact with each other yet still co-operate or interact with each other.

It will be appreciated by persons skilled in the art that numerous variations or modifications may be made to the present technology as shown in the specific implementations without departing from the spirit or scope of the invention as broadly described. For example, any formulas given above are merely representative of procedures that may be used. Functionality may be added or deleted from the block diagrams and operations may be interchanged among functional blocks. Steps may be added or deleted to methods described within the scope of the present technology.

The disclosed implementations are, therefore, to be considered in all respects as illustrative and not limiting or restrictive.

The features described in relation to one or more aspects of the present technology are to be understood as applicable to other aspects of the present technology. More generally, combinations of the steps in the method(s) of the present technology or the features of the system(s) or device(s) of the present technology described elsewhere in this specification, including in the claims, are to be understood as falling within the scope of the disclosure of this specification.

LABEL LIST

| | |
|---|---|
| stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| stimulation circuitry | 124 |
| electrode selection module | 126 |
| measurement circuitry | 128 |
| ground | 130 |
| electrode assembly | 150 |
| biphasic stimulus pulse | 160 |
| ECAP | 170 |
| target nerve fibres | 180 |
| communications channel | 190 |
| external computing device | 192 |
| CLNS system | 300 |
| clinical settings controller | 302 |
| target ECAP controller | 304 |
| dashed box | 308 |
| box | 309 |
| controller | 310 |
| box | 311 |
| stimulator | 312 |
| simulator | 312A |
| simulator | 312B |
| simulator | 312C |
| summing element | 313 |
| measurement circuitry | 318 |
| signal window | 319 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| target ECAP amplitude | 520 |
| ECAP | 600 |
| neural stimulation system | 700 |
| neuromodulation device | 710 |
| remote controller | 720 |
| CST | 730 |
| CI | 740 |
| charger | 750 |
| data flow | 800 |
| neuromodulation device | 804 |
| clinical programming application | 810 |
| clinical data log file | 812 |
| clinical data viewer | 814 |
| Clinical Data Uploader | 816 |
| Database Loader | 822 |
| Database | 824 |
| data analysis web API | 826 |
| analysis module | 832 |
| first stimulus pulse | 810 |
| inter-stimulus interval | 815 |
| stimulus pulse | 820 |
| stimulus pulse | 830 |
| stimulus pulse | 840 |
| stimulus pulse | 850 |
| ECAP | 860 |
| multi - stimset CLNS system | 1000 |
| electrode lead assembly | 1100 |
| electrode arrangement | 1102 |
| reference span | 1104 |
| electrode | 1106 |
| electrode length | 1108 |
| electrode gap | 1110 |
| electrode pitch | 1111 |
| connector section | 1112 |
| electrode | 1114 |
| electrode | 1115 |
| electrode | 1116 |
| electrode | 1118 |
| electrode | 1120 |
| electrode | 1122 |
| start portion | 1150 |
| end portion | 1160 |
| method | 1200 |
| method step | 1202 |
| method step | 1204 |
| method step | 1206 |
| method step | 1208 |
| electrode lead assembly | 1300 |
| Group 1 electrodes | 1302 |
| Group 2 electrodes | 1304 |
| Group 3 electrodes | 1306 |
| first electrode free portion | 1310 |
| first electrode free portion | 1312 |
| second electrode free portion | 1314 |
| electrode lead assembly | 1400 |
| electrode lead assembly | 1500 |
| electrode lead assembly | 1600 |
| outer electrode | 1610 |
| middle electrode | 1620 |
| outer electrode | 1630 |
| electrode | 1702 |
| electrode | 1703 |
| electrode | 1704 |
| electrode | 1705 |
| electrode | 1706 |
| electrode | 1707 |
| amplifier | 1710 |
| current source | 1712 |
| current source | 1714 |
| recording electrode | 1802 |
| reference electrode | 1804 |
| pitch | 1806 |
| electrode lead assembly | 1900 |

1. An electrode lead assembly comprising:
an electrode lead body; and
a plurality of electrodes arranged along a length of the electrode lead body,
the plurality of electrodes comprising a first group of electrodes and a second group of electrodes, each group of electrodes comprising two or more electrodes, wherein adjacent pairs of electrodes in each group of electrodes have a respective electrode pitch,
wherein the first group of electrodes and the second group of electrodes each comprise:
at least one electrode configurable as a stimulus electrode for delivering stimulation to a neural target; and
at least one electrode configurable as a measurement electrode for measuring a response evoked by the delivered stimulation, and
wherein the first group of electrodes is separated from the second group of electrodes by an electrode-free lead portion, the electrode-free lead portion having a length that is greater than each electrode pitch of the first group of electrodes and greater than each electrode pitch of the second group of electrodes.

2. The electrode lead assembly of claim 1, wherein the plurality of electrodes further comprises a third group of electrodes.

3. The electrode lead assembly of claim 2, wherein each of the first group of electrodes, the second group of electrodes and the third group of electrodes comprises three electrodes.

4. The electrode lead assembly of claim 2, wherein each of the first group of electrodes, the second group of electrodes and the third group of electrodes are configurable to provide tripolar stimulation.

5. The electrode lead assembly of claim 2, wherein each of the first group of electrodes, the second group of electrodes and the third group of electrodes comprises at least two electrodes configurable as measurement electrodes.

6. The electrode lead assembly of claim 2, wherein the second group of electrodes is separated from the third group of electrodes by a second electrode-free lead portion, the second electrode-free lead portion having a length that is greater than an electrode pitch of the second group of electrodes and greater than an electrode pitch of the third group of electrodes.

7. The electrode lead assembly of claim 1, wherein the first group of electrodes comprises at least two slim electrodes.

8. The electrode lead assembly of claim 2, wherein at least one of the first group of electrodes, the second group of electrodes or the third group of electrodes comprises at least two slim electrodes.

9. The electrode lead assembly of claim 8, wherein at least one electrode of the at least two slim electrodes has a length of at least 1.5 millimetres.

10. The electrode lead assembly of claim 8, wherein at least one electrode of the at least two slim electrodes has a length no greater than 2 millimetres.

11. The electrode lead assembly of claim 8, wherein the two slim electrodes are adjacent electrodes, and an electrode pitch between the two slim electrodes is equal to or greater than about 2 millimetres and equal to or less than 4 millimetres.

12. The electrode lead assembly of claim 1, wherein each electrode of the plurality of electrodes comprises a ring electrode.

13. The electrode lead assembly of claim 1, wherein the electrode lead assembly is configured to be electrically connected to an implantable stimulation device comprising stimulation circuitry and measurement circuitry.

14. The electrode lead assembly of claim 1, wherein at least two electrodes are configured as measurement electrodes, and the two measurement electrodes have a pitch of 1.5 to 2 times a pitch of a pair of electrodes configured as stimulation electrodes.

15. The electrode lead assembly of claim 1, wherein at least one group of electrodes has adjacent pairs of electrodes with a varying pitch.

16. The electrode lead assembly of claim 15, wherein the plurality of electrodes arranged along the electrode lead body comprise one or more middle electrodes, wherein the pitch for adjacent pairs of electrodes increases for adjacent pairs closer to the one or more middle electrodes.

17. The electrode lead assembly of claim 16, wherein a profile of the pitch variation is substantially linear.

18. The electrode lead assembly of claim 16, wherein a profile of the pitch variation is symmetrical about the one or more middle electrodes.

19. An implantable device for controllably stimulating a neural target, the device configurable to electrically couple to the electrode lead assembly of claim 1, the implantable device comprising:
stimulation circuitry, configurable to provide stimulation energy to one or more electrodes of the electrode lead assembly;
measurement circuitry, configurable to measure a response evoked from the neural target by the stimulation energy and sensed by one or more electrodes of the electrode lead assembly; and
an electrode selection module, configurable to,
select at least one first electrode from the plurality of electrodes of the electrode lead assembly and electrically couple the first electrode to the stimulation circuitry, and
select at least one second electrode from the plurality of electrodes of the electrode lead assembly and electrically couple the second electrode to the measurement circuitry.

20. A system for controllable neural stimulation including an implantable pulse generator and an electrode lead assembly, the electrode lead assembly comprising:
a plurality of electrodes comprising a first group of electrodes and a second group of electrodes, each group of electrodes comprising two or more electrodes, wherein adjacent pairs of electrodes in each group of electrodes have a respective electrode pitch, the first group of electrodes and the second group of electrodes each comprising:
at least one electrode configurable as a stimulus electrode for delivering stimulation to a neural target; and
at least one electrode configurable as a measurement electrode for measuring a response evoked by the delivered stimulation,
wherein the first group of electrodes is separated from the second group of electrodes by an electrode-free lead portion, the electrode-free lead portion having a length that is greater than each electrode pitch of the first group of electrodes and greater than each electrode pitch of the second group of electrodes;
and
the implantable pulse generator comprising:
stimulation circuitry, configurable to provide stimulation energy to one or more electrodes of the electrode lead assembly;
measurement circuitry, configurable to measure a response evoked from the neural target by the stimulation energy and sensed by one or more electrodes of the electrode lead assembly; and
an electrode selection module, configurable to,
select at least one first electrode from the plurality of electrodes of the electrode lead assembly and electrically couple the first electrode to the stimulation circuitry, and
select at least one second electrode from the plurality of electrodes of the electrode lead assembly and electrically couple the second electrode to the measurement circuitry.
